(19) United States
(12) Patent Application Publication
(10) Pub. No.: US 2024/0218026 A1
(43) Pub. Date: Jul. 4, 2024

(54) MODIFIED MENINGOCOCCAL FHBP POLYPEPTIDES

(71) Applicant: GLAXOSMITHKLINE BIOLOGICALS SA, Rixensart (BE)

(72) Inventors: Matthew BOTTOMLEY, Siena (IT); Enrico MALITO, Siena (IT); Manuele MARTINELLI, Siena (IT); Maria SCARSELLI, Siena (IT)

(73) Assignee: GLAXOSMITHKLINE BIOLOGICALS SA, Rixensart (BE)

(21) Appl. No.: 18/440,212

(22) Filed: Feb. 13, 2024

Related U.S. Application Data
(62) Division of application No. 17/245,681, filed on Apr. 30, 2021, now Pat. No. 11,932,671.
Application No. 17/245,681 is a division of application No. 16/458,365, filed on Jul. 1, 2019, now Pat. No. 11,021,522.
Application No. 16/458,365 is a division of application No. 15/120,674, filed on Aug. 22, 2016, now Pat. No. 10,392,424.

(30) Foreign Application Priority Data
Feb. 28, 2014 (EP) 14157399.8
Jul. 17, 2014 (EP) 14177566.8

(51) Int. Cl. C07K 14/22; A61K 39/095; A61K 39/00

(52) U.S. Cl. CPC C07K 14/22; A61K 39/095; A61K 2039/523; A61K 2039/55511; A61K 2039/70

(57) ABSTRACT

Modified meningococcal fHbp polypeptides with increased stability.

This application claims the benefit of European patent applications 14157399.8 (filed Feb. 28, 2014) and 14177566.8 (filed Jul. 17, 2014), the complete contents of both of which are hereby incorporated herein by reference for all purposes.

### TECHNICAL FIELD

This invention is in the field of protein engineering, relating in particular to the meningococcal factor H binding protein (fHbp), which is known to be a useful vaccine immunogen.

### BACKGROUND ART

Neisseria meningitidis is a Gram-negative encapsulated bacterium which colonises the upper respiratory tract of approximately 10% of human population. Conjugate vaccines are available against serogroups A, C, W135 and Y, but the only vaccine which is available for protecting against serogroup B in a two-dose regimen is the BEXSERO™ product which was approved in 2013.

One of the protective immunogens in BEXSERO™ is fHbp, which has also been known as protein ‘741’ (SEQ ID NO: 2536 in ref. 1; SEQ ID 1 herein), ‘NMB1870’, ‘GNA1870’ [2-4, ‘P2086’, ‘LP2086’ or ‘ORF2086’ [5-7]. The 3D structure of this protein is known [8,9], and the protein has two β-barrels connected by a short linker. Many publications have reported on the protective efficacy of this protein in meningococcal vaccines e.g. see references 10-14. The fHbp lipoprotein is expressed in various strains across all serogroups. fHbp sequences have been grouped into three variants [2] (referred to herein as v1, v2 and v3), and it has been found in general that serum raised against a given variant is bactericidal against strains which express that variant, but is not active against strains which express one of the other two variants i.e. there is intra-variant cross-protection, but not inter-variant cross-protection (except for some v2 and v3 cross-reactivity).

To increase inter-family cross-reactivity the fHbp sequence has been engineered to contain specificities for all three variants [15]. Protein engineering has also been used to remove fHbp's interaction with siderophores [16] and with factor H [17-25]. Disruption of the interaction with fH has been reported for all three variants and is postulated to provide a superior vaccine immunogen ×[22,26]. For v2 polypeptides, however, references 23 and 24 report an inherent instability which is also seen in mutants with disrupted fH-binding. The instability appears to arise from the N-terminal β-barrel domain, and reference 23 warns that any substitutions in this barrel might promote instability.

It is an object of the invention to provide further fHbp v2 and v3 mutants, but having enhanced stability.

### DISCLOSURE OF THE INVENTION

Full-length fHbp from strain 2996 in v2 has the
following amino acid sequence (SEQ ID NO: 2):
MNRTAFCCLSLTAALILTACSSGGGGVAADIGAGLADALTAPLDHKDKS
LQSLTLDQSVRKNEKLKLAAQGAEKTYGNGDSLNTGKLKNDKVSREDFI
RQIEVDGQLITLESGEFQIYKQDHSAVVALQIEKINNPDKIDSLINQRS
FLVSGLGGEHTAFNQLPDGKAEYHGKAFSSDDAGGKLTYTIDFAAKQGH
GKIEHLKTPEQNVELAAAELKADEKSHAVILGDTRYGSEEKGTYHLALF
GDRAQEIAGSATVKIGEKVHEIGIAGKQ

The mature lipoprotein lacks the first 19 amino acids of SEQ ID NO: 2 (underlined; provides SEQ ID NO: 4), and the ΔG form of SEQ ID NO: 2 lacks the first 26 amino acids (SEQ ID NO: 5).

Full-length fHbp from strain M1239 in v3 has the
following amino acid sequence (SEQ ID NO: 3):
MNRTAFCCLSLTTALILTACSSGGGGSGGGGVAADIGTGLADALTAPLD
HKDKGLKSLTLEDSIPQNGTLTLSAQGAEKTFKAGDKDNSLNTGKLKND
KISRFDFVQKIEVDGQTITLASGEFQIYKQNHSAVVALQIEKINNPDKT
DSLINQRSFLVSGLGGEHTAFNQLPGGKAEYHGKAFSSDDPNGRLHYSI
DFTKKQGYGRIEHLKTLEQNVELAAAELKADEKSHAVILGDTRYGSEEK
GTYHLALFGDRAQEIAGSATVKIGEKVHEIGIAGKQ

The mature lipoprotein lacks the first 19 amino acids of SEQ ID NO: 3 (underlined; provides SEQ ID NO: 40), and the ΔG form of SEQ ID NO: 3 lacks the first 31 amino acids (SEQ ID NO: 17).

The inventors have identified residues within SEQ ID NO: 2 and SEQ ID NO: 3 which can be modified to increase the polypeptide's stability. These residues are generally present across v2 and v3 sequences and so their modification can provide v2 and v3 fHbp sequences with enhanced stability. Moreover, the inventors have shown that, as well as increasing stability, mutation of these residues can advantageously decrease binding to human factor H (fH). In addition, however, the mutations disclosed herein can be combined with other mutations e.g. to decrease binding to human factor H (fH), for which several mutations are already known in the art.

Thus, in general the invention provides a mutant v2 or v3 fHbp which has increased stability relative to a wild-type fHbp (e.g. relative to SEQ ID NOs: 2 or 3) and which, optionally, has lower affinity for human factor H than a wild-type fHbp (e.g. relative to SEQ ID NOs: 2 or 3). The increase in stability and the optional reduction in fH affinity preferably result from the same mutation(s), but in some embodiments they may be due to the separate effect of combined mutations. Mutant fHbp proteins with both increased stability and reduced fH affinity are preferred.

In a first embodiment the invention provides a polypeptide comprising a mutant fHbp v2 amino acid sequence, wherein: (a) the amino acid sequence has at least k % sequence identity to SEQ ID NO: 5, and/or comprises a fragment of SEQ ID NO: 5; but (b) the amino acid sequence differs from SEQ ID NO: 5 at one or more of the following residues: S32, V33, L39, L41, F69, V100, I113, F122, L123, V124, S125, G126, L127, G128, S151, H239, and/or E240.

Where feature (a) relates to a fragment, the fragment will include at least one of the residues listed in (b), but that residue will differ when compared to that residue in SEQ ID NO: 5. A fragment of (a) will generally be at least 7 amino acids long e.g. 8, 10, 12, 14, 16, 18, 20, 22, 24, 26, 28, 30, 32, 24, 26, 28, 40, 45, 50, 55, 60 contiguous amino acids or more from SEQ ID NO: 5. The fragment will typically include at least one epitope from SEQ ID NO: 5. Epitope identification and mapping is established for fHbp [11; 27-31]. Sharing at least 30 contiguous amino acids with SEQ ID NO: 5 will be typical, and usually a mutant fHbp v2 amino acid sequence will include several (e.g. 2, 3, 4, 5 or more) fragments from SEQ ID NO: 5. Overall, a mutant fHbp v2 amino acid sequence can have at least k % sequence identity to and include several fragments of SEQ ID NO: 5.

The value of k may be selected from 80, 85, 86, 87, 88, 89, 90, 91, 92, 93, 94, 95, 96, 97, 98, 99 or more. It is preferably 90 (i.e. the mutant fHbp v2 amino acid sequence has at least 90% identity to SEQ ID NO: 5) and is more preferably 95.

The polypeptide can, after administration to a host animal, elicit antibodies which can recognise a wild-type meningococcal polypeptide consisting of SEQ ID NO: 4. These antibodies are ideally bactericidal (see below). These antibodies can include some antibodies which do not recognise a v1 or a v3 polypeptide (e.g. a wild-type meningococcal polypeptide consisting of SEQ ID NO: 46 and a wild-type meningococcal polypeptide consisting of SEQ ID NO: 40), although they may also include some antibodies which cross-react with v1 and/or v3 polypeptides.

The polypeptide has, under the same experimental conditions, a higher stability than the same polypeptide but without the sequence difference/s of (b) e.g. higher stability than a wild-type meningococcal polypeptide consisting of SEQ ID NO: 4. The stability enhancement can be assessed using differential scanning calorimetry (DSC) e.g. as discussed in references 32 & 33. DSC has previously been used to assess the stability of v2 fHbp [24]. Suitable conditions for DSC to assess stability can use 20 μM of polypeptide in a buffered solution (e.g. 25 mM Tris) with a pH between 6 and 8 (e.g. 7-7.5) with 100-200 mM NaCl (e.g. 150 mM).

In some embodiments, the polypeptide of the invention is truncated relative to SEQ ID NO: 5. Compared to the wild-type mature sequence, SEQ ID NO: 5 is already truncated at the N-terminus up to and including the poly-glycine sequence (compare SEQ ID NOs: 4 and 5), but SEQ ID NO: 5 can be truncated at the C-terminus and/or further truncated at the N-terminus.

The increase in stability is ideally at least 5° C. e.g. at least 10° C., 15° C., 20° C., 25° C., 30° C., 35° C. or more. These temperatures refer to the increase in thermal transition midpoint (Tm) as assessed by DSC. Wild-type fHbp shows two DSC peaks during unfolding (one for the N-terminal domain and one for the C-terminal domain) and, where a polypeptide of the invention includes both such domains, the increase refers to the stability of the N-terminal domain, which can occur even below 40° C. with wild-type v2 sequences [24] (whereas C-terminal domains can have a Tm of 80° C. or more). Thus the mutant fHbp v2 amino acid sequence of the invention preferably has a N-terminal domain with a Tm of at least 45° C. e.g. ≥50° C., ≥55° C., ≥60° C., ≥65° C., ≥70° C., ≥75° C., or even ≥80° C.

In a second embodiment, the invention provides a polypeptide comprising a mutant fHbp v3 amino acid sequence, wherein: (a) the amino acid sequence has at least j % sequence identity to SEQ ID NO: 17, and/or comprises a fragment of SEQ ID NO: 17; but (b) the amino acid sequence differs from SEQ ID NO: 17 at one or more of the following residues: S32, I33, L39, L41, F72, V103, T116, F125, L126, V127, S128, G129, L130, G131, S154, H242, and/or E243.

Where feature (a) relates to a fragment, the fragment will include at least one of the residues listed in (b), but that residue will differ when compared to that residue in SEQ ID NO: 17. A fragment of (a) will generally be at least 7 amino acids long e.g. 8, 10, 12, 14, 16, 18, 20, 22, 24, 26, 28, 30, 32, 24, 26, 28, 40, 45, 50, 55, 60 contiguous amino acids or more from SEQ ID NO: 17. The fragment will typically include at least one epitope from SEQ ID NO: 17. Epitope identification and mapping is established for fHbp [11; 27-31]. Sharing at least 30 contiguous amino acids with SEQ ID NO: 17 will be typical, and usually a mutant fHbp v3 amino acid sequence will include several (e.g. 2, 3, 4, 5 or more) fragments from SEQ ID NO: 17. Overall, a mutant fHbp v3 amino acid sequence can have at least j % sequence identity to and include several fragments of SEQ ID NO: 17.

The value of j may be selected from 80, 85, 86, 87, 88, 89, 90, 91, 92, 93, 94, 95, 96, 97, 98, 99 or more. It is preferably 90 (i.e. the mutant fHbp v3 amino acid sequence has at least 90% identity to SEQ ID NO: 17) and is more preferably 95.

The polypeptide can, after administration to a host animal, elicit antibodies which can recognise a wild-type meningococcal polypeptide consisting of SEQ ID NO: 40. These antibodies are ideally bactericidal (see below). These antibodies can include some antibodies which do not recognise a v1 or a v2 polypeptide (e.g. a wild-type meningococcal polypeptide consisting of SEQ ID NO: 46 and a wild-type meningococcal polypeptide consisting of SEQ ID NO: 4), although they may also include some antibodies which cross-react with v1 and/or v2 polypeptides.

The polypeptide has, under the same experimental conditions, a higher stability than the same polypeptide but without the sequence difference/s of (b) e.g. higher stability than a wild-type meningococcal polypeptide consisting of SEQ ID NO: 40. The stability enhancement can be assessed using differential scanning calorimetry (DSC) e.g. as discussed in references 32 & 33 DSC has previously been used to assess the stability of v3 fHbp [23]. Suitable conditions for DSC to assess stability can use 20 μM of polypeptide in a buffered solution (e.g. 25 mM Tris) with a pH between 6 and 8 (e.g. 7-7.5) with 100-200 mM NaCl (e.g. 150 mM).

In some embodiments, the polypeptide of the invention is truncated relative to SEQ ID NO: 17. Compared to the wild-type mature sequence, SEQ ID NO: 17 is already truncated at the N-terminus up to and including the poly-glycine sequence (compare SEQ ID NOs: 40 and 17), but SEQ ID NO: 17 can be truncated at the C-terminus and/or further truncated at the N-terminus.

The increase in stability is ideally at least 5° C. e.g. at least 10° C., 15° C., 20° C., 25° C., 30° C., 35° C. or more. These temperatures refer to the increase in thermal transition midpoint (Tm) as assessed by DSC. Wild-type fHbp shows two DSC peaks during unfolding (one for the N-terminal domain and one for the C-terminal domain) and, where a polypeptide of the invention includes both such domains, the increase refers to the stability of the N-terminal domain, which can occur at around 60° C. or less with wild-type v3 sequences [24] (whereas C-terminal domains can have a Tm of 80° C. or more). Thus the mutant fHbp v3 amino acid sequence of the invention preferably has a N-terminal domain with a Tm of at least 65° C. e.g. ≥70° C., ≥75° C., or even ≥80° C.

Mutations relative to SEQ ID NO: 5 Polypeptides of the first embodiment of the invention comprise an amino acid sequence which has at least k % identity to SEQ ID NO: 5, and/or comprise a fragment of SEQ ID NO: 5. In comparison to SEQ ID NO: 5, however, this amino sequence has a modification at one or more of amino acid residues S32, V33, L39, L41, F69, V100, I113, F122, L123, V124, S125, G126, L127, G128, S151, H239, and/or E240 e.g. at 2, 3, 4, 5 or more of these 17 residues. These residues are numbered according to SEQ ID NO: 5; to match the nascent wild-type sequence (SEQ ID NO: 2), the numbering should change +26 (i.e. Ser-32 of SEQ ID NO: 5 is Ser-58 of SEQ ID NO: 2), and to match the mature wild-type sequence (SEQ ID NO: 4) the numbering should change +7 (which also permits easy comparison with ref. 25).

Preferred residues for mutation are 532, V100, L123, V124, S125, G126, L127, G128, H239, and/or E240. Mutations at these residues give proteins having good stability compared to wild-type v2. Within this subset, preferred residues are S32, L123, V124, S125, G126, L127, and/or G128. The most preferred positions are S32, L123, V124, S125, G126, L127, and/or G128, with residues S32 and/or L123 being particularly preferred e.g. S32V and/or L123R. Where one or more of V100, S125, and/or G126 is mutated, it is preferred to mutate also a residue outside this trio.

The specified residue can be deleted, but preferably it is substituted by a different amino acid. For example, Ser-32 can be substituted by any of the other 19 naturally-occurring amino acids. When a substitution is made, the replacement amino acid in some embodiments may be a simple amino acid such as glycine or alanine. In other embodiments, the replacement amino acid is a conservative substitution e.g. it is made within the following four groups: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. In other embodiments the substitution is non-conservative. In some embodiments the substitution does not use alanine.

Preferred substitutions at the specified residues are as follows: S32V; V33C; L39C; L41C; F69C; V100T; I113S; F122C; L123R; V124I; S125G or S125T; G126D; L127I; G128A; S151C; H239R; E240H.

Where the mutant fHbp v2 amino acid sequence includes a substitution at E240, this substitution will not be with alanine if only E240 is mutated, although it can be alanine if a further amino acid listed in (b) is substituted e.g. if residues E240 and H239 are both mutated. Ideally, E240 is not mutated on its own, and so a mutant fHbp v2 amino acid sequence with a substitution at E240 should also include a substitution at a second residue e.g. at both E240 and H239 (see mutants #1 and #11).

Where the mutant fHbp v2 amino acid sequence includes a substitution at F122, this substitution will not be with alanine if only F122 is mutated, although it can be alanine if a further amino acid listed in (b) is substituted e.g. if residues F122 and S151 are both mutated. Ideally, F122 is not mutated on its own, and so a mutant fHbp v2 amino acid sequence with a substitution at F122 should also include a substitution at a second residue. When F122 is substituted it is preferred that S151 is also substituted e.g. both are substituted with cysteine, to permit formation of a disulfide bridge (see mutant #10).

Where the mutant fHbp v2 amino acid sequence includes a substitution at L123, this substitution will not be with alanine if only L123 is mutated, although it can be alanine if a further amino acid listed in (b) is substituted e.g. if residues L123 and S32 are both mutated. If L123 is mutated on its own, substitution with arginine is preferred (e.g. see mutant #4). In some embodiments, however, L123 is not mutated on its own, and so a mutant fHbp v2 amino acid sequence with a substitution at L123 can also include a substitution at a second residue. When L123 is substituted it can be preferred that: (i) S32 is also substituted, as seen in mutant #3, and optionally S125 is also substituted, as seen in mutants #20 and #22; or (ii) one or more of residues 124-128 is/arc also substituted e.g. as seen in mutant #12.

Where the mutant fHbp v2 amino acid sequence includes a substitution at V124, it is preferred that this substitution will not be with alanine if only V124 is mutated, although it can be alanine if a further amino acid listed in (b) is substituted e.g. if one or more of residues 123-128 are also mutated. If V124 is mutated on its own, substitution with isoleucine is preferred. Ideally, however, V124 is not mutated on its own, and so a mutant fHbp v2 amino acid sequence with a substitution at V124 should also include a substitution at a second residue. When V124 is substituted it is preferred that one or more of residues 124-128 is/are also substituted e.g. as seen in mutant #12.

Where the mutant fHbp v2 amino acid sequence includes a substitution at L127, it is preferred that this substitution will not be with alanine if only L127 is mutated, although it can be alanine if a further amino acid listed in (b) is substituted e.g. if one or more of residues 123-128 are also mutated. If L127 is mutated on its own, substitution with isoleucine is preferred. Ideally, however, L127 is not mutated on its own, and so a mutant fHbp v2 amino acid sequence with a substitution at L127 should also include a substitution at a second residue. When L127 is substituted it is preferred that one or more of residues 124-128 is/are also substituted e.g. as seen in mutant #12.

Where the mutant fHbp v2 amino acid sequence includes a substitution at S32, it is preferred that this substitution will not be with alanine if only S32 is mutated, although it can be alanine if a further amino acid listed in (b) is substituted e.g. if residues L123 and S32 are both mutated. If S32 is mutated on its own, substitution with valine is preferred. Ideally, however, S32 is not mutated on its own, and so a mutant fHbp v2 amino acid sequence with a substitution at S32 should also include a substitution at a second residue. When S32 is substituted it is preferred that (i) L123 is also substituted e.g. as seen in mutant #3, and optionally S125 is also substituted, as seen in mutants #20 and #22; or (ii) S125 is also substituted e.g. as seen in mutants #19 and #21.

Where the mutant fHbp v2 amino acid sequence includes a substitution at I113, this substitution will not be with alanine if only I113 is mutated, although it can be alanine if a further amino acid listed in (b) is substituted. If I113 is mutated on its own, substitution with serine is preferred e.g. as seen in mutant #7.

Where the mutant fHbp v2 amino acid sequence includes a substitution at V33, this preferably is not with isoleucine. Where the mutant fHbp v2 amino acid sequence includes a substitution at I113, this preferably is not with threonine or with alanine. Where the mutant fHbp v2 amino acid sequence includes a substitution at S151, this preferably is not with phenylalanine. Where the mutant fHbp v2 amino acid sequence includes a substitution at both H239 and E240, this preferably is not to R239 and Q240.

Where more than one substitution is made, these may be selected from groups 2A to 2O as follows:
2A: residues 239 and 240 e.g. mutant #1.
2B: residues 32 and 123 e.g. mutant #3.
2C: residues 125 and 126 e.g. mutant #5.
2D: residues 100, 125 and 126 e.g. mutant #6.
2E: residues 33 and 39 e.g. mutant #8.
2F: residues 41 and 69 e.g. mutant #9.
2G: residues 122 and 151 e.g. mutant #10.
2H: residues 100, 125, 126, 239 and 240 e.g. mutant #11.
2I: residues 32 and 125 e.g. mutants #19 and #21.
2J: residues 32, 123 and 125 e.g. mutants #20 and #22.
2K: residues 33 and 39, both substituted by Cys e.g. mutant #8.
2L: residues 41 and 69, both substituted by Cys e.g. mutant #9.
2M: residues 122 and 151, both substituted by Cys e.g. mutant #10.
2N: residues 123, 124, 125, 126, 127 and 128 e.g. mutant #12.
2O: residues 32, 123, 124, 125, 126, 127 and 128.

Thus, for example, if residue 239 is to be substituted then a preferred second residue for substitution is 240 (i.e. group 2A); moreover, residues 100, 125 and 126 might also be modified (i.e. group 2H, which is a combination of groups 2A and 2D). Within groups 2A to 2N & 20, preferred substitutions at the specified positions are those listed above. For groups 2K, 2L & 2M, the intention is to introduce a disulfide bridge. Within groups 2A to 2N, preferred mutants are 2A, 2B, 2C, 2D, 2I, 2J, and 2N. More preferred are 2C, 2I, and 2N, with 2N being particularly preferred. Group 2B provides the most preferred mutations, and in particular S32V and L123R (e.g. SEQ ID NOs: 20 and 45). Group 2O is another preferred set of mutations, which combines 2B, 2C and three further mutations (e.g. to give SEQ ID NO: 58).

The amino acid residues noted for mutation in a v2 sequence are numbered relative to SEQ ID NO: 5 which is from strain 2996. The corresponding amino acid residues in a v2 fHbp from any other strain can be readily identified by sequence alignment e.g. being the amino acid which, when aligned to SEQ ID NO: 5 using a pairwise alignment algorithm (e.g. the Needleman-Wunsch global alignment algorithm, as detailed below), aligns with the amino acid mentioned herein. Often the amino acid will be the same as seen in SEQ ID NO: 5 (e.g. residue 32 will be serine), but the alignment will easily identify if this is not the case.

In addition to the mutation(s) noted above, which aim to increase stability, a polypeptide of the invention can include one or more further mutation(s) e.g. to disrupt the polypeptide's interaction with siderophores or, more preferably, to disrupt the polypeptide's ability to bind to fH.

References 19 and 25 report that the interaction between fH and v2 fHbp can be disrupted by mutations at residues R80, D211, E218, E248, T220+H222 (double mutation), and G236. Numbered according to SEQ ID NO: 5, these residues are R73, D203, E210, E240, T213+H215, and G228. Of these positions, polypeptides mutated at D203, E210 or T213+H215 are preferred because reference 25 reports no impairment of important epitopes in these mutants. The specific substitutions studied in reference 25 were R73A, D203A, E210A, T213A+H215A, G228I, and E240A; these substitutions are suitable for use according to the invention.

Reference 24 reports that the interaction between fH and v2 fHbp can be disrupted by mutations at residues R145, S193, F194, L195, A265, E267, K268, V272, I273, L274, E283, T286, H288, F292, T304, and E313 and E283+T304 (double mutation). Numbered according to SEQ ID NO: 5, these residues are R73, S121, F122, L123, A192, E194, K195, V199, I200, L201, E210, T213, H215, F219, T231, and E240 and E210+T231. Four of these overlap with reference 25 (E210, T213, H215, E240). The specific substitutions studied in reference 24 used alanine (except for A265P and T304E), and these substitutions are suitable for use according to the invention.

Reference 24 reports that certain substitutions in v2 can increase affinity for fH, and these should be avoided if the intention is to disrupt binding to fH e.g. E85 in SEQ ID NO: 5 (residue 157 in ref. 24).

Residues which interact with siderophores can be mutated, using the guidance in references 16 and 34 e.g. by aligning SEQ ID NO: 5 herein with SEQ ID NO: 4 of reference 16 to identify residues which can interact with siderophores e.g. with catecholates, hydroxamates or carboxylates.

Further residues which can be mutated include, but are not limited to, S23, L24, D30, Q31, R34, D95, and/or L102 e.g. using the mutations suggested in reference 35.

The polypeptide of the first embodiment can comprise any of SEQ ID NOs: 18 to 36. Similarly, taking into account the ‘ΔG’ mutation (i.e. truncation of the nascent N-terminus up to and including the native poly-Gly sequence), the polypeptide of the first embodiment can comprise any of SEQ ID NOs: 18 to 36 excluding amino acids 1-26 thereof. For example, the polypeptide of the first embodiment can comprise SEQ ID NO: 45, or can comprise SEQ ID NO: 58.

Considering the possibility of further point mutations (e.g. to disrupt interactions with siderophores and/or fH) the polypeptide of the first embodiment can comprise any of SEQ ID NOs: 18 to 36 (or any of SEQ ID NOs: 18 to 36 excluding amino acids 1-26 thereof, such as SEQ ID NO: 45) but modified by up to 5 single amino acid changes (i.e. 1, 2, 3, 4 or 5 single amino acid substitutions, deletions and/or insertions) provided that the modified sequence can, after administration to a host animal, elicit antibodies which bind to a meningococcal fHbp polypeptide consisting of the amino acid sequence of SEQ ID NO: 46. Such amino acid changes should not reverse the mutations in these sequences relative to the wild-type sequence e.g. SEQ ID NO: 45 should not be mutated at residue V32 or R123.

The invention also provides a polypeptide comprising a fHbp v2 amino acid sequence, wherein the v2 amino acid sequence is identical to a v2 wild-type amino acid sequence except for a mutation at the amino acid position corresponding to Leu-123 of SEQ ID NO: 5, provided that the mutation is not a substitution to alanine (e.g. wherein the mutation is a substitution to arginine). For instance, the polypeptide can comprise SEQ ID NO: 5, but with a mutation (other than L123A) at L123.

SEQ ID NOs: 59 and 60 are two further examples of v2 mutants, namely the mature form of mutants #3 & #4 for strain 8047.

### Mutations Relative to SEQ ID NO: 17

Polypeptides of the second embodiment of the invention comprise an amino acid sequence which has at least j % identity to SEQ ID NO: 17, and/or comprise a fragment of SEQ ID NO: 17. In comparison to SEQ ID NO: 17, however, this amino sequence has a modification at one or more of amino acid residues S32, I33, L39, L41, F72, V103, T116, F125, L126, V127, S128, G129, L130, G131, S154, H242, and/or E243 e.g. at 2, 3, 4, 5 or more of these 17 residues. These residues are numbered according to SEQ ID NO: 17; to match the nascent wild-type sequence (SEQ ID NO: 3), the numbering should change +31 (i.e. Ser-32 of SEQ ID NO: 17 is Ser-63 of SEQ ID NO: 3), and to match the mature wild-type sequence (SEQ ID NO: 40) the numbering should change +12.

Preferred residues for mutation are S32, V103, L126, V127, S128, G129, L130, G131, H242, and/or E243. Within this subset, preferred residues are S32, L126, V127, S128, G129, L130, and/or G131. The most preferred positions are S32, L126, V127, S128, G129, L130, and/or G131, with residues S32 and/or L126 being particularly preferred e.g. S32V and/or L126R.

The specified residue can be deleted, but preferably it is substituted by a different amino acid. For example, Ser-32 can be substituted by any of the other 19 naturally-occurring amino acids. When a substitution is made, the replacement amino acid in some embodiments may be a simple amino acid such as glycine or alanine. In other embodiments, the replacement amino acid is a conservative substitution e.g. it is made within the following four groups: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. In other embodiments the substitution is non-conservative. In some embodiments the substitution does not use alanine.

Preferred substitutions at the specified residues are as follows: S32V; I33C; L39C; L41C; F72C; V103T; T116S; F125C; L126R; V127I; S128G or S128T; G129D; L130I; G131A; S154C; H242R; E243H.

Where the mutant fHbp v3 amino acid sequence includes a substitution at E243, this substitution will not be with alanine if only E243 is mutated, although it can be alanine if a further amino acid listed in (b) is substituted e.g. if residues E243 and H242 are both mutated. Ideally, E243 is not mutated on its own, and so a mutant fHbp v3 amino acid sequence with a substitution at E243 should also include a substitution at a second residue e.g. at both E243 and H242.

Where the mutant fHbp v3 amino acid sequence includes a substitution at F125, this substitution will not be with alanine if only F125 is mutated, although it can be alanine if a further amino acid listed in (b) is substituted e.g. if residues F125 and S154 are both mutated. Ideally, F125 is not mutated on its own, and so a mutant fHbp v3 amino acid sequence with a substitution at F125 should also include a substitution at a second residue. When F125 is substituted it is preferred that S154 is also substituted e.g. both are substituted with cysteine, to permit formation of a disulfide bridge.

Where the mutant fHbp v3 amino acid sequence includes a substitution at L126, this substitution will not be with alanine if only L126 is mutated, although it can be alanine if a further amino acid listed in (b) is substituted e.g. if residues L126 and S32 are both mutated. If L126 is mutated on its own, substitution with arginine is preferred. In some embodiments, however, L126 is not mutated on its own, and so a mutant fHbp v3 amino acid sequence with a substitution at L126 can also include a substitution at a second residue. When L126 is substituted it can be preferred that: (i) S32 is also substituted, and optionally S128 is also substituted; or (ii) one or more of residues 127-131 is/are also substituted.

Where the mutant fHbp v3 amino acid sequence includes a substitution at V127, it is preferred that this substitution will not be with alanine if only V127 is mutated, although it can be alanine if a further amino acid listed in (b) is substituted e.g. if one or more of residues 126-131 are also mutated. If V127 is mutated on its own, substitution with isoleucine is preferred. Ideally, however, V127 is not mutated on its own, and so a mutant fHbp v3 amino acid sequence with a substitution at V127 should also include a substitution at a second residue. When V127 is substituted it is preferred that one or more of residues 127-131 is/are also substituted.

Where the mutant fHbp v3 amino acid sequence includes a substitution at L130, it is preferred that this substitution will not be with alanine if only L130 is mutated, although it can be alanine if a further amino acid listed in (b) is substituted e.g. if one or more of residues 126-131 are also mutated. If L130 is mutated on its own, substitution with isoleucine is preferred. Ideally, however, L130 is not mutated on its own, and so a mutant fHbp v3 amino acid sequence with a substitution at L130 should also include a substitution at a second residue. When L130 is substituted it is preferred that one or more of residues 127-131 is/are also substituted.

Where the mutant fHbp v3 amino acid sequence includes a substitution at S32, it is preferred that this substitution will not be with alanine if only S32 is mutated, although it can be alanine if a further amino acid listed in (b) is substituted e.g. if one or more of residues if residues L126 and S32 are both mutated. If S32 is mutated on its own, substitution with valine is preferred. Ideally, however, S32 is not mutated on its own, and so a mutant fHbp v3 amino acid sequence with a substitution at S32 should also include a substitution at a second residue. When S32 is substituted it is preferred that (i) L 126 is also substituted, and optionally S128 us also substituted, or (ii) S128 is also substituted.

Where the mutant fHbp v3 amino acid sequence includes a substitution at T113, this substitution will not be with alanine if only T113 is mutated, although it can be alanine if a further amino acid listed in (b) is substituted. If T113 is mutated on its own, substitution with serine is preferred.

Where the mutant fHbp v3 amino acid sequence includes a substitution at 133, this preferably is not with valine. Where the mutant fHbp v3 amino acid sequence includes a substitution at T116, this preferably is not with isoleucine. Where the mutant fHbp v3 amino acid sequence includes a substitution at G129, this preferably is not with serine. Where the mutant fHbp v3 amino acid sequence includes a substitution at both H242 and E243, this preferably is not to R242 and Q243.

Where more than one substitution is made, these may be selected from groups 3A to 30 as follows:
3A: residues 242 and 243.
3B: residues 32 and 126.
3C: residues 128 and 129.
3D: residues 103, 128 and 129.
3E: residues 33 and 39.
3F: residues 41 and 72.
3G: residues 125 and 154.
3H: residues 103, 128, 129, 242 and 243.
3I: residues 32 and 128.
3J: residues 32, 126 and 128.
3K: residues 33 and 39, both substituted by Cys.
3L: residues 41 and 72, both substituted by Cys.
3M: residues 125 and 154, both substituted by Cys.
3N: residues 126, 127, 128, 129, 130 and 131.
3O: residues 32, 126, 127, 128, 129, 130 and 131.

Thus, for example, if residue 242 is to be substituted then a preferred second residue for substitution is 243 (i.e. group 3A); moreover, residues 103, 128 and 129 might also be modified (i.e. group 3H, which is a combination of groups 3A and 3D). Within groups 3A to 3N & 30, preferred substitutions at the specified positions are those listed above. For groups 3K, 3L & 3M, the intention is to introduce a disulfide bridge. Within groups 3A to 3N, preferred mutants are 3A, 3B, 3C, 3D, 3I, 3J, and 3N. More preferred are 3C, 3I, and 3N, with 3N being particularly preferred. Group 3B provides the most preferred mutations, and in particular S32V and L126R (e.g. comprising SEQ ID NO: 44). Group 3O is another preferred mutation, which combines 3B, 3C and three further mutations (e.g. to give SEQ ID NO: 61). Mutation L126R alone provides SEQ ID NO: 53.

The amino acid residues noted for mutation in a v3 sequence are numbered relative to SEQ ID NO: 17 which is from strain M1239. The corresponding amino acid residues in a v3 fHbp from any other strain can be readily identified by sequence alignment e.g. being the amino acid which, when aligned to SEQ ID NO: 17 using a pairwise alignment algorithm (e.g. the Needleman-Wunsch global alignment algorithm, as detailed below), aligns with the amino acid mentioned herein. Often the amino acid will be the same as seen in SEQ ID NO: 17 (e.g. residue 32 will be serine), but the alignment will easily identify if this is not the case.

In addition to the mutation(s) noted above, which aim to increase stability, a polypeptide of the invention can include one or more further mutation(s) e.g. to disrupt the polypeptide's interaction with siderophores or, more preferably, to disrupt the polypeptide's ability to bind to fH.

Reference 24 reports that the interaction between fH and v3 fHbp can be disrupted by mutations at residues Q107, I147, L156, A157, L195, V196, V272, E283, T286, T304, V311, E313 and E283+T304 (double mutation). Numbered according to SEQ ID NO: 17, these residues are: Q35, 178, L87, A88, L126, V127, V202, E213, T216, T234, V241, E243 and E213+T234. The specific substitutions studied in reference 24 used alanine (except for A157E and T231E), and these substitutions are suitable for use according to the invention. Residues T216 and E243 are also reported in reference 23. Reference 36 reports that the interaction between fH and v3 fHbp can be disrupted by mutations at residues H288 and G318 (H218 and G248 numbered according to SEQ ID NO: 17), and these substitutions are suitable for use according to the invention e.g. H218R, G248D.

Ref 24 reports that certain substitutions in v3 can increase affinity for fH, and these should be avoided if the intention is to disrupt binding to fH e.g. P44 in SEQ ID NO: 17 (residue 106 in ref. 24)

Residues which interact with siderophores can be mutated, using the guidance in references 16 and 34 e.g. by aligning SEQ ID NO: 17 herein with SEQ ID NO: 4 of reference 16 to identify residues which can interact with siderophores e.g. with catecholates, hydroxamates or carboxylates.

The polypeptide of the second embodiment can comprise any of SEQ ID NOs: 41 to 44. Considering the possibility of further point mutations (e.g. to disrupt interactions with siderophores and/or fH) the polypeptide of the first embodiment can comprise any of SEQ ID NOs: 41 to 44 but modified by up to 5 single amino acid changes (i.e. 1, 2, 3, 4 or 5 single amino acid substitutions, deletions and/or insertions) provided that the modified sequence can, after administration to a host animal, elicit antibodies which bind to a meningococcal fHbp polypeptide consisting of the amino acid sequence of SEQ ID NO: 40. Such amino acid changes should not reverse the mutations in these sequences relative to the wild-type sequence e.g. SEQ ID NO: 44 should not be mutated at residue V32 or R126.

The invention also provides a polypeptide comprising a fHbp v3 amino acid sequence, wherein the v3 amino acid sequence is identical to a v3 wild-type amino acid sequence except for a mutation at the amino acid position corresponding to Leu-126 of SEQ ID NO: 17, provided that the mutation is not a substitution to alanine (e.g. wherein the mutation is a substitution to arginine). For instance, the polypeptide can comprise SEQ ID NO: 17, but with a mutation (other than L126A) at L126.

### Polypeptides

Polypeptides of the invention can be prepared by various means e.g. by chemical synthesis (at least in part), by digesting longer polypeptides using proteases, by translation from RNA, by purification from cell culture (e.g. from recombinant expression or from N. meningitidis culture), etc. Heterologous expression in an E. coli host is a preferred expression route.

Polypeptides of the invention are ideally at least 100 amino acids long e.g. 150aa, 175aa, 200aa, 225aa, or longer. They include a mutant fHbp v2 and/or v3 amino acid sequence, and the mutant fHbp v2 or v3 amino acid sequence should similarly be at least 100 amino acids long e.g. 150aa, 175aa, 200aa, 225aa, or longer.

The fHbp is naturally a lipoprotein in N. meningitidis. It has also been found to be lipidated when expressed in E. coli with its native leader sequence or with heterologous leader sequences. Polypeptides of the invention may have a N-terminus cysteine residue, which may be lipidated e.g. comprising a palmitoyl group, usually forming tripalmitoyl-S-glyceryl-cysteine. In other embodiments the polypeptides are not lipidated.

Polypeptides are preferably prepared in substantially pure or substantially isolated form (i.e. substantially free from other Neisserial or host cell polypeptides). In general, the polypeptides are provided in a non-naturally occurring environment e.g. they are separated from their naturally-occurring environment. In certain embodiments, the polypeptide is present in a composition that is enriched for the polypeptide as compared to a starting material. Thus purified polypeptide is provided, whereby purified means that the polypeptide is present in a composition that is substantially free of other expressed polypeptides, whereby substantially free is meant that more than 50% (e.g. ≥75%, ≥80%, ≥90%, ≥95%, or ≥99%) of total polypeptide in the composition is a polypeptide of the invention.

Polypeptides can take various forms (e.g. native, fusions, glycosylated, non-glycosylated, lipidated, disulfide bridges, etc.).

SEQ ID NOs 4, 5, 17 and 40 do not include a N-terminus methionine. If a polypeptide of the invention is produced by translation in a biological host then a start codon is required, which will provide a N-terminus methionine in most hosts. Thus a polypeptide of the invention will, at least at a nascent stage, include a methionine residue upstream of said SEQ ID NO sequence.

Cleavage of nascent sequences means that the mutant fHbp v2 or v3 amino acid sequence might itself provide the polypeptide's N-terminus. In other embodiments, however, a polypeptide of the invention can include a N-terminal sequence upstream of the mutant fHbp v2 or v3 amino acid sequence. In some embodiments the polypeptide has a single methionine at the N-terminus immediately followed by the mutant fHbp v2 or v3 amino acid sequence; in other embodiments a longer upstream sequence may be used. Such an upstream sequence may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. a histidine tag i.e. His, where n=4, 5, 6, 7, 8, 9, 10 or more). Other suitable N-terminal amino acid sequences will be apparent to those skilled in the art e.g. the native upstream sequences present in SEQ ID NO: 2 or SEQ ID NO: 3.

A polypeptide of the invention may also include amino acids downstream of the final amino acid of the mutant fHbp v2 or v3 amino acid sequence. Such C-terminal extensions may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising a histidine tag i.e. His, where n=4, 5, 6, 7, 8, 9, 10 or more), or sequences which enhance polypeptide stability. Other suitable C-terminal amino acid sequences will be apparent to those skilled in the art.

In some embodiments, the invention excludes polypeptides which include a histidine tag (cf. refs. 24 & 25), and in particular a hexahistidine tag at the C-terminus.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

Polypeptides of the invention may be attached or immobilised to a solid support.

Polypeptides of the invention may comprise a detectable label e.g. a radioactive label, a fluorescent label, or a biotin label. This is particularly useful in immunoassay techniques.

As disclosed in reference 164, fHbp can be split
into three domains, referred to as A, B and C.
Taking SEQ ID NO: 1, the three domains are (A)
1-119, (B) 120-183 and (C) 184-274:
MNRTAFCCLSLTTALILTACSSGGGGVAADIGAGLADALTAPLDHKDKG
LQSLTLDQSVRKNEKLKLAAQGAEKTYGNGDSLNTGKLKNDKVSRFDFI
RQIEVDGQLITLESGEFQVYKQSHSALTAFQTEQIQDSEHSGKMVAKRQ
FRIGDIAGEHTSFDKLPEGGRATYRGTAFGSDDAGGKLTYTIDFAAKQG
NGKIEHLKSPELNVDLAAADIKPDGKRHAVISGSVLYNQAEKGSYSLGI
FGGKAQEVAGSAEVKTVNGIRHIGLAAKQ

The mature form of domain ‘A’, from Cys-20 at its N-terminus to Lys-119, is called ‘Aₘₐₜᵤᵣₑ’.

Multiple fHbp sequences are known and these can readily be aligned using standard methods. By such alignments the skilled person can identify (a) domains ‘A’ (and ‘Aₘₐₜᵤᵣₑ’), ‘B’ and ‘C’ in any given fHbp sequence by comparison to the coordinates in the MC58 sequence, and (b) single residues in multiple fHbp sequences e.g. for identifying substitutions. For ease of reference, however, the domains are defined below:
Domain ‘A’ in a given fHbp sequence is the fragment of that sequence which, when aligned to SEQ ID NO: 1 using a pairwise alignment algorithm, starts with the amino acid aligned to Met-1 of SEQ ID NO: 1 and ends with the amino acid aligned to Lys-119 of SEQ ID NO: 1.
Domain ‘Aₘₐₜᵤᵣₑ’ in a given fHbp sequence is the fragment of that sequence which, when aligned to SEQ ID NO: 1 using a pairwise alignment algorithm, starts with the amino acid aligned to Cys-20 of SEQ ID NO: 1 and ends with the amino acid aligned to Lys-119 of SEQ ID NO: 1.
Domain ‘B’ in a given fHbp sequence is the fragment of that sequence which, when aligned to SEQ ID NO: 1 using a pairwise alignment algorithm, starts with the amino acid aligned to Gln-120 of SEQ ID NO: 1 and ends with the amino acid aligned to Gly-183 of SEQ ID NO: 1.
Domain ‘C’ in a given fHbp sequence is the fragment of that sequence which, when aligned to SEQ ID NO: 1 using a pairwise alignment algorithm, starts with the amino acid aligned to Lys-184 of SEQ ID NO: 1 and ends with the amino acid aligned to Gln-274 of SEQ ID NO: 1.

The preferred pairwise alignment algorithm for defining the domains is the Needleman-Wunsch global alignment algorithm [158], using default parameters (e.g. with Gap opening penalty=10.0, and with Gap extension penalty=0.5, using the EBLOSUM62 scoring matrix). This algorithm is conveniently implemented in the needle tool in the EMBOSS package [159].

In some embodiments, a mutant fHbp v2 or v3 amino acid sequence of the invention is truncated to remove its domain A. In general, however, it is preferred that the mutant fHbp v2 or v3 amino acid sequence should include both a N-terminal β-barrel and a C-terminal β-barrel.

In some embodiments, a polypeptide comprises an amino acid sequence as described above, except that up to 10 amino acids (i.e. 1, 2, 3, 4, 5, 6, 7, 8, 9 or 10) at the N-terminus and/or up to 10 amino acids (i.e. 1, 2, 3, 4, 5, 6, 7, 8, 9 or 10) at the C-terminus are deleted.

Polypeptides of the invention typically consist of an artificial amino acid sequence, namely a sequence which is not present in any naturally-occurring meningococci.

Affinity for factor H can be quantitatively assessed using surface plasmon resonance e.g. as disclosed in references 18 and 21-24 with immobilised human fH. Mutations which provide an affinity reduction (i.e. an increase in the dissociation constant, K_{D}) of at least 10-fold, and ideally at least 100-fold, is preferred (when measured under the same experimental conditions relative to the same polypeptide but without the mutation).

### Nucleic Acids

The invention provides nucleic acid encoding a polypeptide of the invention as defined above.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acids of the invention can take various forms e.g. single-stranded, double-stranded, vectors, primers, probes, labelled, unlabelled, etc.

Nucleic acids of the invention are preferably in isolated or substantially isolated form.

The term “nucleic acid” includes DNA and RNA, and also their analogues, such as those containing modified backbones, and also peptide nucleic acids (PNA), etc.

Nucleic acid according to the invention may be labelled e.g. with a radioactive or fluorescent label.

The invention also provides vectors (such as plasmids) comprising nucleotide sequences of the invention (e.g. cloning or expression vectors, such as those suitable for nucleic acid immunisation) and host cells transformed with such vectors.

### Bactericidal Responses

Preferred polypeptides of the invention can elicit antibody responses that are bactericidal against meningococci. Bactericidal antibody responses are conveniently measured in mice and are a standard indicator of vaccine efficacy (e.g. see end-note 14 of ref. 37; also ref. 38).

Polypeptides of the first embodiment invention can preferably elicit an antibody response which is bactericidal against a N. meningitidis strain which expresses a v2 fHbp sequence e.g. one or more of strains 961-5945, 2996, 96217, 312294, 11327, a22, gb013 (=M01-240013), e32, m1090, m4287, 860800, 599, 95N477, 90-18311, e11, m986, m2671, 1000, m1096, m3279, bz232, dk353, m3697, ngh38, and/or L93/4286. Bactericidal responses can for instance be assessed against var2 strain M2091 (ATCC 13091).

Preferred polypeptides of the first embodiment invention can elicit antibodies in a mouse which are bactericidal against strain M2091 in a serum bactericidal assay.

Polypeptides of the second embodiment invention can preferably elicit an antibody response which is bactericidal against a N. meningitidis strain which expresses a v3 fHbp sequence e.g. one or more of strains M1239, 16889, gb355 (=M01-240355), m3369, m3813, ngp165. Bactericidal responses can for instance be assessed against var3 strain M01-240355, which is a Neisseria MLST reference strains (id 19265 in ref. 39) which has been fully sequenced (see EMBL ID CP002422 [40])

Preferred polypeptides of the second embodiment invention can elicit antibodies in a mouse which are bactericidal against strain M01-240355 in a serum bactericidal assay.

For example, an immunogenic composition comprising these polypeptides can provide a serum bactericidal titer of ≥1:4 using the Goldschneider assay with human complement [41-43], and/or providing a serum bactericidal titer of ≥1:128 using baby rabbit complement.

### Immunisation

Polypeptides of the invention may be used as the active ingredient of immunogenic compositions, and so the invention provides an immunogenic composition (e.g. a vaccine) comprising a polypeptide of the invention.

The invention also provides a method for raising an antibody response in a mammal, comprising administering an immunogenic composition of the invention to the mammal. The antibody response is preferably a protective and/or bactericidal antibody response. The invention also provides polypeptides of the invention for use in such methods.

The invention also provides a method for protecting a mammal against a Neisserial (e.g. meningococcal) infection, comprising administering to the mammal an immunogenic composition of the invention.

The invention provides polypeptides of the invention for use as medicaments (e.g. as immunogenic compositions or as vaccines) or as diagnostic reagents. It also provides the use of nucleic acid or polypeptide of the invention in the manufacture of a medicament for preventing Neisserial (e.g. meningococcal) infection in a mammal.

The mammal is preferably a human. The human may be an adult or, preferably, a child. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

The uses and methods are particularly useful for preventing/treating diseases including, but not limited to, meningitis (particularly bacterial, such as meningococcal, meningitis) and bacteremia. For instance, they are suitable for active immunisation of individuals against invasive meningococcal disease caused by N. meningitidis (for example in serogroup B).

Efficacy of therapeutic treatment can be tested by monitoring Neisserial infection after administration of the composition of the invention. Efficacy of prophylactic treatment can be tested by monitoring immune responses against fHbp after administration of the composition. Immunogenicity of compositions of the invention can be determined by administering them to test subjects (e.g. children 12-16 months age, or animal models) and then determining standard parameters including serum bactericidal antibodies (SBA) and ELISA titres (GMT). These immune responses will generally be determined around 4 weeks after administration of the composition, and compared to values determined before administration of the composition. A SBA increase of at least 4-fold or 8-fold is preferred. Where more than one dose of the composition is administered, more than one post-administration determination may be made.

Preferred compositions of the invention can confer an antibody titre in a patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is about 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

The immunogenic composition of the invention will generally include a pharmaceutically acceptable carrier, which can be any substance that does not itself induce the production of antibodies harmful to the patient receiving the composition, and which can be administered without undue toxicity. Pharmaceutically acceptable carriers can include liquids such as water, saline, glycerol and ethanol. Auxiliary substances, such as wetting or emulsifying agents, pH buffering substances, and the like, can also be present in such vehicles. A thorough discussion of suitable carriers is available in ref. 44.

Neisserial infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be prepared for topical administration e.g. as an ointment, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. Compositions suitable for parenteral injection are most preferred.

The composition is preferably sterile. It is preferably pyrogen-free. It is preferably buffered e.g. at between pH 6 and pH 8, generally around pH 7. Where a composition comprises an aluminium hydroxide salt, it is preferred to use a histidine buffer [45]. Compositions of the invention may be isotonic with respect to humans.

Immunogenic compositions comprise an immunologically effective amount of immunogen, as well as any other of other specified components, as needed. By ‘immunologically effective amount’, it is meant that the administration of that amount to an individual, either in a single dose or as part of a series, is effective for treatment or prevention. The term “prevention” means that the progression of the disease is reduced and/or eliminated, or that the onset of the disease is eliminated. For example, the immune system of a subject may be primed (e.g by vaccination) to trigger an immune response and repel infection such that the onset of the disease is eliminated. A vaccinated subject may thus get infected, but is better able to repel the infection than a control subject. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Dosage treatment may be a single dose schedule or a multiple dose schedule (e.g. including booster doses). The composition may be administered in conjunction with other immunoregulatory agents.

Adjuvants which may be used in compositions of the invention include, but are not limited to insoluble metal salts, oil-in-water emulsions (e.g. MF59 or AS03, both containing squalene), saponins, non-toxic derivatives of LPS (such as monophosphoryl lipid A or 3-O-deacylated MPL), immunostimulatory oligonucleotides, detoxified bacterial ADP-ribosylating toxins, microparticles, liposomes, imidazoquinolones, or mixtures thereof. Other substances that act as immunostimulating agents are disclosed in chapter 7 of ref. 46.

The use of an aluminium hydroxide and/or aluminium phosphate adjuvant is particularly preferred, and polypeptides are generally adsorbed to these salts. These salts include oxyhydroxides and hydroxyphosphates (e.g. see chapters 8 & 9 of ref. 46). The salts can take any suitable form (e.g. gel, crystalline, amorphous, etc.).

### Further Antigenic Components

Compositions of the invention include mutant v2 and/or v3 fHbp sequence. It is useful if the composition should not include complex or undefined mixtures of antigens e.g. it is preferred not to include outer membrane vesicles in the composition. Polypeptides of the invention are preferably expressed recombinantly in a heterologous host and then purified.

As well as including a fHbp polypeptide, a composition of the invention may also include one or more further neisserial immunogen(s), as a vaccine which targets more than one immunogen per bacterium decreases the possibility of selecting escape mutants. Thus a composition can include a second polypeptide that, when administered to a mammal, elicits an antibody response that is bactericidal against meningococcus. The second polypeptide can be a meningococcal fHbp, but will often not be a fHbp e.g. it may be a NHBA sequence, a NadA sequence, etc.

Any such further neisserial immunogen may be present as a separate polypeptide to the mutant v2 or v3 fHbp of the invention or may be present as a fusion polypeptide with the modified fHbp. For instance, fusion of meningococcal 936 polypeptide and fHbp polypeptides is known [55,56]. Fusion proteins comprising SEQ ID NO: 44 and/or SEQ ID NO: 45 are particularly preferred, optionally wherein SEQ ID NO: 44 and/or 45 is/are modified by up to 5 single amino acid changes (i.e. 1, 2, 3, 4 or 5 single amino acid substitutions, deletions and/or insertions) as described elsewhere herein.

A composition of the invention may include a NHBA antigen. The NHBA antigen was included in the published genome sequence for meningococcal serogroup B strain MC58 [47] as gene NMB2132 (GenBank accession number GI:7227388; SEQ ID NO: 6 herein). The sequences of NHBA antigen from many strains have been published since then. For example, allelic forms of NHBA can be seen in FIGS. 5 and 15 of reference 48, and in example 13 and FIG. 21 of reference 1 (SEQ IDs 3179 to 3184 therein). Various immunogenic fragments of the NHBA antigen have also been reported. Preferred 287 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 6; and/or (b) comprising a fragment of at least ‘n’ consecutive amino acids of SEQ ID NO: 6, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 6. The most useful NHBA antigens of the invention can elicit antibodies which, after administration to a subject, can bind to a meningococcal polypeptide consisting of amino acid sequence SEQ ID NO: 6. Advantageous NHBA antigens for use with the invention can elicit bactericidal anti-meningococcal antibodies after administration to a subject.

A composition of the invention may include a NadA antigen. The NadA antigen was included in the published genome sequence for meningococcal serogroup B strain MC58 [47] as gene NMB1994 (GenBank accession number GI:7227256; SEQ ID NO: 7 herein). The sequences of NadA antigen from many strains have been published since then, and the protein's activity as a Neisserial adhesin has been well documented. Various immunogenic fragments of NadA have also been reported. Preferred NadA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7; and/or (b) comprising a fragment of at least ‘n’ consecutive amino acids of SEQ ID NO: 7, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 7. The most useful NadA antigens of the invention can elicit antibodies which, after administration to a subject, can bind to a meningococcal polypeptide consisting of amino acid sequence SEQ ID NO: 7. Advantageous NadA antigens for use with the invention can elicit bactericidal anti-meningococcal antibodies after administration to a subject. SEQ ID NO: 15 is one such fragment.

A composition of the invention may include a NspA antigen. The NspA antigen was included in the published genome sequence for meningococcal serogroup B strain MC58 [47] as gene NMB0663 (GenBank accession number GI:7225888; SEQ ID NO: 8 herein). The antigen was previously known from references 49 & 50. The sequences of NspA antigen from many strains have been published since then. Various immunogenic fragments of NspA have also been reported. Preferred NspA antigens for use with the invention comprise n amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) comprising a fragment of at least ‘n’ consecutive amino acids of SEQ ID NO: 8, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 8. The most useful NspA antigens of the invention can elicit antibodies which, after administration to a subject, can bind to a meningococcal polypeptide consisting of amino acid sequence SEQ ID NO: 8. Advantageous NspA antigens for use with the invention can elicit bactericidal anti-meningococcal antibodies after administration to a subject.

Compositions of the invention may include a meningococcal HmbR antigen. The full-length HmbR sequence was included in the published genome sequence for meningococcal serogroup B strain MC58 [47] as gene NMB1668 (SEQ ID NO: 9 herein). The invention can use a polypeptide that comprises a full-length HmbR sequence, but it will often use a polypeptide that comprises a partial HmbR sequence. Thus in some embodiments a HmbR sequence used according to the invention may comprise an amino acid sequence having at least i % sequence identity to SEQ ID NO: 9, where the value of i is 50, 60, 70, 80, 90, 95, 99 or more. In other embodiments a HmbR sequence used according to the invention may comprise a fragment of at least j consecutive amino acids from SEQ ID NO: 9, where the value of j is 7, 8, 10, 12, 14, 16, 18, 20, 25, 30, 35, 40, 50, 60, 70, 80, 90, 100, 150, 200, 250 or more. In other embodiments a HmbR sequence used according to the invention may comprise an amino acid sequence (i) having at least i % sequence identity to SEQ ID NO: 9 and/or (ii) comprising a fragment of at least j consecutive amino acids from SEQ ID NO: 9. Preferred fragments of j amino acids comprise an epitope from SEQ ID NO: 9. Such epitopes will usually comprise amino acids that are located on the surface of HmbR. Useful epitopes include those with amino acids involved in HmbR's binding to haemoglobin, as antibodies that bind to these epitopes can block the ability of a bacterium to bind to host haemoglobin. The topology of HmbR, and its critical functional residues, were investigated in reference 51. The most useful HmbR antigens of the invention can elicit antibodies which, after administration to a subject, can bind to a meningococcal polypeptide consisting of amino acid sequence SEQ ID NO: 9. Advantageous HmbR antigens for use with the invention can elicit bactericidal anti-meningococcal antibodies after administration to a subject.

A composition of the invention may include a NhhA antigen. The NhhA antigen was included in the published genome sequence for meningococcal serogroup B strain MC58 [47] as gene NMB0992 (GenBank accession number GI:7226232; SEQ ID NO: 10 herein). The sequences of NhhA antigen from many strains have been published since e.g. refs 48 & 52, and various immunogenic fragments of NhhA have been reported. It is also known as Hsf. Preferred NhhA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO: 10, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 10. The most useful NhhA antigens of the invention can elicit antibodies which, after administration to a subject, can bind to a meningococcal polypeptide consisting of amino acid sequence SEQ ID NO: 10. Advantageous NhhA antigens for use with the invention can elicit bactericidal anti-meningococcal antibodies after administration to a subject.

A composition of the invention may include an App antigen. The App antigen was included in the published genome sequence for meningococcal serogroup B strain MC58 [47] as gene NMB1985 (GenBank accession number GI:7227246; SEQ ID NO: 11 herein). The sequences of App antigen from many strains have been published since then. Various immunogenic fragments of App have also been reported. Preferred App antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 11. The most useful App antigens of the invention can elicit antibodies which, after administration to a subject, can bind to a meningococcal polypeptide consisting of amino acid sequence SEQ ID NO: 11. Advantageous App antigens for use with the invention can elicit bactericidal anti-meningococcal antibodies after administration to a subject.

A composition of the invention may include an Omp85 antigen. The Omp85 antigen was included in the published genome sequence for meningococcal serogroup B strain MC58 [47] as gene NMB0182 (GenBank accession number GI:7225401; SEQ ID NO: 12 herein). The sequences of Omp85 antigen from many strains have been published since then. Further information on Omp85 can be found in references 53 and 54. Various immunogenic fragments of Omp85 have also been reported. Preferred Omp85 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 12. The most useful Omp85 antigens of the invention can elicit antibodies which, after administration to a subject, can bind to a meningococcal polypeptide consisting of amino acid sequence SEQ ID NO: 12. Advantageous Omp85 antigens for use with the invention can elicit bactericidal anti-meningococcal antibodies after administration to a subject.

A composition of the invention may include a 936 antigen. The 936 antigen was included in the published genome sequence for meningococcal serogroup B strain MC58 [47] as gene NMB2091 (SEQ ID NO: 13 herein). Preferred 936 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13; and/or (b) comprising a fragment of at least ‘n’ consecutive amino acids of SEQ ID NO: 13, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 13. The most useful 936 antigens of the invention can elicit antibodies which, after administration to a subject, can bind to a meningococcal polypeptide consisting of amino acid sequence SEQ ID NO: 13. The 936 antigen is a good fusion partner for fHbp (e.g. see references 55 & 56).

A composition may comprise: a polypeptide comprising SEQ ID NO: 14; a polypeptide comprising SEQ ID NO: 15; and a polypeptide of the invention comprising a mutant fHbp v2 amino acid sequence and SEQ ID NO: 13 (cf. refs. 55 & 56).

A composition may comprise: a polypeptide comprising SEQ ID NO: 14; a polypeptide comprising SEQ ID NO: 15; and a polypeptide of the invention comprising a mutant fHbp v3 amino acid sequence and SEQ ID NO: 13 (cf. refs. 55 & 56).

In some embodiments, a polypeptide of the invention is combined with a further meningococcal fHbp sequence. In particular, a v2 polypeptide can be combined with a v1 and/or a v3 polypeptide to increase the spectrum of strain coverage [162]. Thus a composition can comprise: (i) a polypeptide of the invention comprising a mutant fHbp v2 amino acid sequence; and (ii) a v1 fHbp polypeptide and/or a v3 fHbp polypeptide. In other embodiments, a polypeptide of the invention can comprise (i) a mutant fHbp v2 amino acid sequence and (ii) a v1 fHbp amino acid sequence and/or a v3 fHbp amino acid sequence. Thus the v1 and/or v3 sequences can be combined with the mutant v2 sequence as separate entities in a composition, or within a fusion polypeptide.

Similarly, a v3 polypeptide can be combined with a v1 and/or a v2 polypeptide to increase the spectrum of strain coverage [162]. Thus a composition can comprise: (i) a polypeptide of the invention comprising a mutant fHbp v3 amino acid sequence; and (ii) a v1 fHbp polypeptide and/or a v2 fHbp polypeptide. In other embodiments, a polypeptide of the invention can comprise (i) a mutant fHbp v3 amino acid sequence and (ii) a v1 fHbp amino acid sequence and/or a v2 fHbp amino acid sequence. Thus the v1 and/or v2 sequences can be combined with the mutant v3 sequence as separate entities in a composition, or within a fusion polypeptide.

Moreover, mutant v2 and v3 polypeptides can be combined with each other to increase strain coverage. Thus a composition can comprise: (i) a polypeptide of the invention comprising a mutant fHbp v2 amino acid sequence; (ii) a polypeptide of the invention comprising a mutant fHbp v3 amino acid sequence; and (iii) a fHbp v1 polypeptide. In other embodiments, a polypeptide of the invention can comprise (i) a mutant fHbp v2 amino acid sequence (ii) a mutant v3 fHbp amino acid sequence and (iii) a fHbp v1 amino acid sequence. Thus the mutant v2 and v3 sequences can be combined with a v1 sequence as separate entities in a composition, or within a fusion polypeptide. The v1 sequence can be a wild-type sequence or a mutant sequence.

A v1 fHbp can comprise (a) an amino acid sequence which has at least k % identity to SEQ ID NO: 16, and/or (b) a fragment of SEQ ID NO: 16. Information about ‘k’ and fragments are given above. The fragment will typically include at least one epitope from SEQ ID NO: 16, and the v1 fHbp polypeptide will include at least one epitope which is not present in the v2 or v3 amino acid sequence of the invention, such that antibodies elicited by the v1 fHbp can recognise v1 strains. Ideally, the v1 fHbp can elicit antibodies which are bactericidal against v1 strains e.g. against strain MC58 (available from the ATCC as ‘BAA-335’). The v1 fHbp can include an amino acid mutation which disrupts its ability to bind to fH.

A v2 fHbp can comprise (a) an amino acid sequence which has at least k % identity to SEQ ID NO: 5, and/or (b) a fragment of SEQ ID NO: 5. Information about ‘k’ and fragments are given above. The fragment will typically include at least one epitope from SEQ ID NO: 5, and the v2 fHbp polypeptide will include at least one epitope which is not present in the v3 amino acid sequence of the invention, such that antibodies elicited by the v2 fHbp can recognise v2 strains. Ideally, the v2 fHbp can elicit antibodies which are bactericidal against v2 strains e.g. against strain M2091 (ATCC 13091). The v2 fHbp can be a polypeptide of the first embodiment.

A v3 fHbp can comprise (a) an amino acid sequence which has at least k % identity to SEQ ID NO: 17, and/or (b) a fragment of SEQ ID NO: 17. Information about ‘k’ and fragments are given above. The fragment will typically include at least one epitope from SEQ ID NO: 17, and the v3 fHbp polypeptide will include at least one epitope which is not present in the v2 amino acid sequence of the invention, such that antibodies elicited by the v3 fHbp can recognise v3 strains. Ideally, the v3 fHbp can elicit antibodies which are bactericidal against v3 strains e.g. against strain M01-240355. The v3 fHbp can be a polypeptide of the second embodiment.

Thus, for instance, the invention provides a polypeptide comprising, within a single polypeptide chain, each of: (i) a fHbp v1 amino acid sequence; (ii) a mutant fHbp v2 amino acid sequence; and (iii) a mutant fHbp v3 amino acid sequence. The polypeptide can, after administration to a host animal, elicit antibodies which bind to each of: a meningococcal fHbp polypeptide consisting of the amino acid sequence of SEQ ID NO: 46; a meningococcal fHbp polypeptide consisting of the amino acid sequence of SEQ ID NO: 4; and a meningococcal fHbp polypeptide consisting of the amino acid sequence of SEQ ID NO: 40. The sequence of (i) can comprise an amino acid sequence which has at least k % identity to SEQ ID NO: 16. The sequence of (ii) can comprise an amino acid sequence which has at least k % identity to SEQ ID NO: 5, but differing from SEQ ID NO: 5 at one or more of the following residues: S32, V33, L39, L41, F69, V100, I113, F122, L123, V124, S125, G126, L127, G128, S151, H239, and/or E240. The sequence of (iii) can comprise an amino acid sequence which has at least k % identity to SEQ ID NO: 17, but differing from SEQ ID NO: 17 at one or more of the following residues: S32, I33, L39, L41, F72, V103, T116, F125, L126, V127, S128, G129, L130, G131, S154, H242, and/or E243. In a preferred embodiment: the sequence of (i) comprises SEQ ID NO: 16, optionally modified by up to 5 single amino acid changes (i.e. 1, 2, 3, 4 or 5 single amino acid substitutions, deletions and/or insertions); the sequence of (ii) comprises SEQ ID NO: 45, optionally modified by up to 5 single amino acid changes (i.e. 1, 2, 3, 4 or 5 single amino acid substitutions, deletions and/or insertions), provided such amino acid changes do not reverse the mutations in these sequences relative to the wild-type sequence; and the sequence of (iii) comprises SEQ ID NO: 44, optionally modified by up to 5 single amino acid changes (i.e. 1, 2, 3, 4 or 5 single amino acid substitutions, deletions and/or insertions e.g. to give SEQ ID NO: 53), provided such amino acid changes do not reverse the mutations in these sequences relative to the wild-type sequence. Amino acid sequences (i), (ii) and (iii) can be arranged in any order from N- to C-terminus in the polypeptide, but are preferably in the order (ii), then (iii), then (i). For instance, the invention provides a polypeptide of formula -A-B-C- wherein: A comprises SEQ ID NO: 45, optionally modified by up to 3 single amino acid substitutions; B comprises SEQ ID NO: 44, optionally modified by up to 3 single amino acid substitutions; and C comprises SEQ ID NO: 16, optionally modified by up to 3 single amino acid substitutions (e.g. substitution(s) to to disrupt binding to fH). A preferred C comprises SEQ ID NO: 49, where residue Arg-34 is mutated to Ser as reported for the ‘R41 S’ mutation in ref. 21.

A particularly preferred polypeptide comprises amino acid sequence SEQ ID NO: 47. This sequence includes, from N-terminus to C-terminus: the mutant v2 (SEQ ID NO: 45); the mutant v3 (SEQ ID NO: 44); and the mutant v1 (SEQ ID NO: 49). Between these three mutant fHbp sequences there is in each case a linker sequence, SEQ ID NO: 50. In one embodiment, the polypeptide comprises amino acid sequence SEQ ID NO: 48, which has a N-terminus methionine, then SEQ ID NO: 37, and then SEQ ID NO: 47.

SEQ ID NO: 47 (alone, or within SEQ ID NO: 48) can optionally be modified by up to 5 single amino acid changes (i.e. 1, 2, 3, 4 or 5 single amino acid substitutions, deletions and/or insertions), provided such amino acid changes do not reverse the mutations in the v1, v2, and v3 sequences relative to the wild-type sequence i.e. amino acid residues V32, R123, V285, R379, and S543 of SEQ ID NO: 47 should not be mutated to S32, L123, S285, L379, and R543. In one exceptional embodiment, however, V285 can revert to S285 and/or V32 can revert to S32.

The mutant fusion can ideally elicit antibodies which bind to each of: a wild-type v1 meningococcal fHbp polypeptide consisting of amino acid sequence SEQ ID NO: 46; a wild-type v2 meningococcal fHbp polypeptide consisting of amino acid sequence SEQ ID NO: 4; and a wild-type v3 meningococcal fHbp polypeptide consisting of amino acid sequence SEQ ID NO: 40.

In addition to Neisserial polypeptide antigens, the composition may include antigens for immunising against other diseases or infections. For example, the composition may include one or more of the following further antigens:
a saccharide antigen from N. meningitidis serogroup A, C, W135 and/or Y, such as the saccharide disclosed in ref. 57 from serogroup C (see also ref. 58) or in ref. 59.
a saccharide antigen from Streptococcus pneumoniae [e.g. 60, 61, 62].
an antigen from hepatitis A virus, such as inactivated virus [e.g. 63, 64].
an antigen from hepatitis B virus, such as the surface and/or core antigens [e.g. 64, 65], a diphtheria antigen, such as a diphtheria toxoid [e.g. chapter 3 of ref. 66] e.g. the CRM₁₉₇ mutant [e.g. 67].
a tetanus antigen, such as a tetanus toxoid (e.g. chapter 4 of ref. 66).
an antigen from Bordetella pertussis, such as pertussis holotoxin (PT) and filamentous haemagglutinin (FHA) from B. pertussis, optionally also in combination with pertactin and/or agglutinogens 2 and 3 (e.g. refs. 68 & 69).
a saccharide antigen from Haemophilus influenzae B [e.g. 58].
polio antigen(s) [e.g. 70, 71] such as IPV.
measles, mumps and/or rubella antigens (e.g. chapters 9, 10 & 11 of ref. 66).
influenza antigen(s) (e.g. chapter 19 of ref. 66), such as the haemagglutinin and/or neuraminidase surface proteins.
an antigen from Moraxella catarrhalis [e.g. 72].
an protein antigen from Streptococcus agalactiae (group B streptococcus) [e.g. 73, 74].
a saccharide antigen from Streptococcus agalactiae (group B streptococcus).
an antigen from Streptococcus pyogenes (group A streptococcus) [e.g. 74, 75, 76].
an antigen from Staphylococcus aureus [e.g. 77].

The composition may comprise one or more of these further antigens.

Toxic protein antigens may be detoxified where necessary (e.g. detoxification of pertussis toxin by chemical and/or genetic means [69]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates are discussed in more detail below.

Antigens in the composition will typically be present at a concentration of at least 1 μg/ml each. In general, the concentration of any given antigen will be sufficient to elicit an immune response against that antigen.

Immunogenic compositions of the invention may be used therapeutically (i.e. to treat an existing infection) or prophylactically (i.e. to prevent future infection).

As an alternative to using proteins antigens in the immunogenic compositions of the invention, nucleic acid (which could be RNA, such as a self-replicating RNA, or DNA, such as a plasmid) encoding the antigen may be used.

In some embodiments a composition of the invention comprises in addition to the fHbp sequence, conjugated capsular saccharide antigens from 1, 2, 3 or 4 of meningococcus serogroups A, C, W135 and Y. In other embodiments a composition of the invention comprises in addition to the fHbp sequence, at least one conjugated pneumococcal capsular saccharide antigen.

### Meningococcus Serogroups Y, W135, C and A

Current serogroup C vaccines (Menjugate™ [57,78], Meningitec™ and NeisVac-C™) include conjugated saccharides. Menjugate™ and Meningitec™ have oligosaccharide antigens conjugated to a CRM₁₉₇ carrier, whereas NeisVac-C™ uses the complete polysaccharide (de-O-acetylated) conjugated to a tetanus toxoid carrier. The Menactra™ vaccine contains conjugated capsular saccharide antigens from each of serogroups Y, W135, C and A.

Compositions of the present invention may include capsular saccharide antigens from one or more of meningococcus serogroups Y, W135, C and A, wherein the antigens are conjugated to carrier protein(s) and/or are oligosaccharides. For example, the composition may include a capsular saccharide antigen from: serogroup C; serogroups A and C; serogroups A, C and W135; serogroups A, C and Y; serogroups C, W135 and Y; or from all four of serogroups A, C, W135 and Y.

A typical quantity of each meningococcal saccharide antigen per dose is between 1 μg and 20 μg e.g. about 1 μg, about 2.5 μg, about 4 μg, about 5 μg, or about 10 μg (expressed as saccharide).

Where a mixture comprises capsular saccharides from both serogroups A and C, the ratio (w/w) of MenA saccharide:MenC saccharide may be greater than 1 (e.g. 2:1, 3:1, 4:1, 5:1, 10:1 or higher). Where a mixture comprises capsular saccharides from serogroup Y and one or both of serogroups C and W135, the ratio (w/w) of MenY saccharide:MenW135 saccharide may be greater than 1 (e.g. 2:1, 3:1, 4:1, 5:1, 10:1 or higher) and/or that the ratio (w/w) of MenY saccharide:MenC saccharide may be less than 1 (e.g. 1:2, 1:3, 1:4, 1:5, or lower). Preferred ratios (w/w) for saccharides from serogroups A:C:W135:Y are: 1:1:1:1; 1:1:1:2; 2:1:1:1; 4:2:1:1; 8:4:2:1; 4:2:1:2; 8:4:1:2; 4:2:2:1; 2:2:1:1; 4:4:2:1; 2:2:1:2; 4:4:1:2; and 2:2:2:1. Preferred ratios (w/w) for saccharides from serogroups C:W135:Y are: 1:1:1; 1:1:2; 1:1:1; 2:1:1; 4:2:1; 2:1:2; 4:1:2; 2:2:1; and 2:1:1. Using a substantially equal mass of each saccharide is preferred.

Capsular saccharides may be used in the form of oligosaccharides. These are conveniently formed by fragmentation of purified capsular polysaccharide (e.g. by hydrolysis), which will usually be followed by purification of the fragments of the desired size.

Fragmentation of polysaccharides is preferably performed to give a final average degree of polymerisation (DP) in the oligosaccharide of less than 30 (e.g. between 10 and 20, preferably around 10 for serogroup A; between 15 and 25 for serogroups W135 and Y, preferably around 15-20; between 12 and 22 for serogroup C; etc.). DP can conveniently be measured by ion exchange chromatography or by colorimetric assays [79].

If hydrolysis is performed, the hydrolysate will generally be sized in order to remove short-length oligosaccharides [58]. This can be achieved in various ways, such as ultrafiltration followed by ion-exchange chromatography. Oligosaccharides with a degree of polymerisation of less than or equal to about 6 are preferably removed for serogroup A, and those less than around 4 are preferably removed for serogroups W135 and Y.

Preferred MenC saccharide antigens are disclosed in reference 78, as used in Menjugate™.

The saccharide antigen may be chemically modified. This is particularly useful for reducing hydrolysis for serogroup A [80]. De-O-acetylation of meningococcal saccharides can be performed. For oligosaccharides, modification may take place before or after depolymerisation.

Where a composition of the invention includes a MenA saccharide antigen, the antigen is preferably a modified saccharide in which one or more of the hydroxyl groups on the native saccharide has/have been replaced by a blocking group [80]. This modification improves resistance to hydrolysis.

### Covalent Conjugation

Capsular saccharides in compositions of the invention will usually be conjugated to carrier protein(s). In general, conjugation enhances the immunogenicity of saccharides as it converts them from T-independent antigens to T-dependent antigens, thus allowing priming for immunological memory. Conjugation is particularly useful for paediatric vaccines and is a well known technique.

Typical carrier proteins are bacterial toxins, such as diphtheria or tetanus toxins, or toxoids or mutants thereof. The CRM₁₉₇ diphtheria toxin mutant [81] is useful, and is the carrier in the PREVNAR™ product. Other suitable carrier proteins include the N. meningitidis outer membrane protein complex [82], synthetic peptides [83,84], heat shock proteins [85,86], pertussis proteins [87,88], cytokines [89], lymphokines [89], hormones [89], growth factors [89], artificial proteins comprising multiple human CD4⁺ T cell epitopes from various pathogen-derived antigens [90] such as N19 [91], protein D from H. influenzae [92-94], pneumolysin [95] or its non-toxic derivatives [96], pneumococcal surface protein PspA [97], iron-uptake proteins [98], toxin A or B from C. difficile [99], recombinant P. aeruginosa exoprotein A (rEPA) [100], etc.

Any suitable conjugation reaction can be used, with any suitable linker where necessary.

The saccharide will typically be activated or functionalised prior to conjugation. Activation may involve, for example, cyanylating reagents such as CDAP (e.g. 1-cyano-4-dimethylamino pyridinium tetrafluoroborate [101,102, etc.]). Other suitable techniques use carbodiimides, hydrazides, active esters, norborane, p-nitrobenzoic acid, N-hydroxysuccinimide, S-NHS, EDC, TSTU, etc.

Linkages via a linker group may be made using any known procedure, for example, the procedures described in references 103 and 104. One type of linkage involves reductive amination of the polysaccharide, coupling the resulting amino group with one end of an adipic acid linker group, and then coupling a protein to the other end of the adipic acid linker group [105,106]. Other linkers include B-propionamido [107], nitrophenyl-ethylamine [108], haloacyl halides [109], glycosidic linkages [110], 6-aminocaproic acid [111], ADH [112], C₄ to C₁₂ moieties [113] etc. As an alternative to using a linker, direct linkage can be used. Direct linkages to the protein may comprise oxidation of the polysaccharide followed by reductive amination with the protein, as described in, for example, references 114 and 115.

A process involving the introduction of amino groups into the saccharide (e.g. by replacing terminal ═O groups with —NH₂) followed by derivatisation with an adipic diester (e.g. adipic acid N-hydroxysuccinimido diester) and reaction with carrier protein is preferred. Another preferred reaction uses CDAP activation with a protein D carrier e.g. for MenA or MenC.

### Outer Membrane Vesicles

Some compositions of the invention do not include complex or undefined mixtures of antigens, which are typical characteristics of OMVs. However, the invention can be used in conjunction with OMVs, as fHbp has been found to enhance their efficacy [4], in particular by over-expressing the polypeptides of the invention in the strains used for OMV preparation. See also below.

This approach may be used in general to improve preparations of N. meningitidis serogroup B microvesicles [116], ‘native OMVs’ [117], blebs or outer membrane vesicles [e.g. refs. 118 to 123, etc.]. These may be prepared from bacteria which have been genetically manipulated [124-127] e.g. to increase immunogenicity (e.g. hyper-express immunogens), to reduce toxicity, to inhibit capsular polysaccharide synthesis, to down-regulate PorA expression, etc. They may be prepared from hyperblebbing strains [128-131]. Vesicles from a non-pathogenic Neisseria may be included [132]. OMVs may be prepared without the use of detergents [133,134]. They may express non-Neisserial proteins on their surface [135]. They may be LPS-depleted. They may be mixed with recombinant antigens [118,136]. Vesicles from bacteria with different class I outer membrane protein subtypes may be used e.g. six different subtypes [137,138] using two different genetically-engineered vesicle populations each displaying three subtypes, or nine different subtypes using three different genetically-engineered vesicle populations each displaying three subtypes, etc. Useful subtypes include: P1.7,16; P1.5-1,2-2; P1.19,15-1; P1.5-2,10; P1.12-1,13; P1.7-2,4; P1.22,14; P1.7-1,1; P1.18-1,3,6.

### Host Cells

The invention provides a bacterium which expresses a polypeptide of the invention. The bacterium may be a meningococcus or an E. coli. The bacterium may constitutively express the polypeptide, but in some embodiments expression may be under the control of an inducible promoter. The bacterium may hyper-express the polypeptide (cf. ref. 139). Expression of the polypeptide is ideally not phase variable.

The invention also provides outer membrane vesicles prepared from a bacterium of the invention (particularly from a meningococcus). It also provides a process for producing vesicles from a bacterium of the invention. Vesicles prepared from these strains preferably include the polypeptide of the invention, which should be in an immunoaccessible form in the vesicles i.e. an antibody which can bind to purified polypeptide of the invention should also be able to bind to the polypeptide which is present in the vesicles.

These outer membrane vesicles include any proteoliposomic vesicle obtained by disruption of or blebbing from a meningococcal outer membrane to form vesicles therefrom that include protein components of the outer membrane. Thus the term includes OMVs (sometimes referred to as ‘blebs’), microvesicles (MVs [116]) and ‘native OMVs’ (‘NOMVs’ [117]).

MVs and NOMVs are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 130 & 131 describe Neisseria with high MV production.

OMVs are prepared artificially from bacteria, and may be prepared using detergent treatment (e.g. with deoxycholate), or by non-detergent means (e.g. see reference 134). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [140 & 141] being preferred for treating Neisseria) at a pH sufficiently high not to precipitate the detergent [142]. Other techniques may be performed substantially in the absence of detergent [134] using techniques such as sonication, homogenisation, microfluidisation, cavitation, osmotic shock, grinding, French press, blending, etc. Methods using no or low detergent can retain useful antigens such as NspA and fHbp [134]. Thus a method may use an OMV extraction buffer with about 0.5% deoxycholate or lower e.g. about 0.2%, about 0.1%, <0.05% or zero.

A useful process for OMV preparation is described in reference 143 and involves ultrafiltration on crude OMVs, rather than instead of high speed centrifugation. The process may involve a step of ultracentrifugation after the ultrafiltration takes place. OMVs can also be purified using the two stage size filtration process described in ref. 154.

Vesicles for use with the invention can be prepared from any meningococcal strain. The vesicles will usually be from a serogroup B strain, but it is possible to prepare them from serogroups other than B (e.g. reference 142 discloses a process for serogroup A), such as A, C, W135 or Y. The strain may be of any serotype (e.g. 1, 2a, 2b, 4, 14, 15, 16, etc.), any serosubtype, and any immunotype (e.g. L1; L2; L3; L3,3,7; L10; etc.). The meningococci may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3.

Bacteria of the invention may, in addition to encoding a polypeptide of the invention, have one or more further modifications. For instance, they may have a modified fur gene [144]. Expression of nspA expression may be up-regulated with concomitant porA and cps knockout. Further knockout mutants of N. meningitidis for OMV production are disclosed e.g. in reference 150. Reference 145 discloses the construction of vesicles from strains modified to express six different PorA subtypes. Mutant Neisseria with low endotoxin levels, achieved by knockout of enzymes involved in LPS biosynthesis, may also be used [146,147]. Mutant Neisseria engineered to reduce or switch off expression of at least one gene involved in rendering toxic the lipid A portion of LPS, in particular of lpxl1 gene, can be used with the invention [148]. Similarly, mutant Neisseria engineered to reduce or switch off expression of at least one gene involved in the capsular polysaccharide synthesis or export, in particular of synX and/or ctrA genes can be used with the invention. These or others mutants can all be used with the invention.

Thus a strain used with the invention may in some embodiments express more than one PorA subtype. 6-valent and 9-valent PorA strains have previously been constructed. The strain may express 2, 3, 4, 5, 6, 7, 8 or 9 of PorA subtypes: P1.7,16; P1.5-1,2-2; P1.19,15-1; P1.5-2,10; P1.12-1,13; P1.7-2,4; P1.22,14; P1.7-1,1 and/or P1.18-1,3,6. In other embodiments a strain may have been down-regulated for PorA expression e.g. in which the amount of PorA has been reduced by at least 20% (e.g. ≥30%, ≥40%, ≥50%, ≥60%, ≥70%, ≥80%, ≥90%, ≥95%, etc.), or even knocked out, relative to wild-type levels (e.g. relative to strain H44/76).

In some embodiments a strain may hyper-express (relative to the corresponding wild-type strain) certain proteins. For instance, strains may hyper-express NspA, protein 287 [118], fHbp [139](including fHbp of the invention), TbpA and/or TbpB [136], Cu,Zn-superoxide dismutase, HmbR, etc.

A gene encoding a polypeptide of the invention may be integrated into the bacterial chromosome or may be present in episomal form e.g. within a plasmid.

Advantageously for vesicle production, a meningococcus may be genetically engineered to ensure that expression of the polypeptide is not subject to phase variation. Methods for reducing or eliminating phase variability of gene expression in meningococcus are disclosed in reference 149. For example, a gene may be placed under the control of a constitutive or inducible promoter, or by removing or replacing the DNA motif which is responsible for its phase variability.

In some embodiments a strain may include one or more of the knockout and/or hyper-expression mutations disclosed in references 122, 124, 128, and 150. For instance, following the guidance and nomenclature in these four documents, useful genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB; (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/or TbpB; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB; or (d) CtrA, CtrB, CtrD, FrpB, OpA, OpC, PilC, PorB, SiaD, SynA, SynB, SynX and/or SynC.

Where a mutant strain is used, in some embodiments it may have one or more, or all, of the following characteristics: (i) down-regulated or knocked-out LgtB and/or GalE to truncate the meningococcal LOS; (ii) up-regulated TbpA; (iii) up-regulated NhhA; (iv) up-regulated Omp85; (v) up-regulated LbpA; (vi) up-regulated NspA; (vii) knocked-out PorA; (viii) down-regulated or knocked-out FrpB; (ix) down-regulated or knocked-out Opa; (x) down-regulated or knocked-out Opc; (xii) deleted cps gene complex. A truncated LOS can be one that does not include a sialyl-lacto-N-neotetraose epitope e.g. it might be a galactose-deficient LOS. The LOS may have no a chain.

Depending on the meningococcal strain used for preparing the vesicles, they may or may not include the strain's native fHbp antigen [151].

In one preferred embodiment, a meningococcus does not express a functional MltA protein. As discussed in refs. 152 & 153, knockout of MltA (the membrane-bound lytic transglycosylase, also known as GNA33) in meningococcus provides bacteria which spontaneously release large amounts of membrane vesicles into culture medium, from which they can be readily purified. For instance, the vesicles can be purified using the two stage size filtration process of ref. 154, comprising: (i) a first filtration step in which vesicles are separated from the bacteria based on their different sizes, with the vesicles passing into the filtrate; and (ii) a second filtration step in which the vesicles are retained in the retentate. The MltA mutation (down-regulation or knockout) has been used in ‘GMMA’ vaccines [155], and can conveniently be combined with further down regulation or knockout of in particular of at least one gene involved in rendering toxic the lipid A portion of LPS, particularly of lpxl1 and/or of at least one gene involved in the capsular polysaccharide synthesis or export, particularly of synX and/or ctrA genes. GMMA (Generalized Modules for Membrane Antigens) are genetically detoxified OMV that are produced from meningococcal strains that have been engineered to release GMMA with reduced reactogenicity and increased immunogenicity. GMMA induce less proinflammatory cytokines than OMV when tested in the monocyte activation test (MAT).

A preferred meningococcal strain for a ‘GMMA’ vaccine using this approach expresses a mutant v2 fHbp and/or a mutant v3 fHbp of the invention, and expression can be driven by strong promoters. Vesicles released by this strain include the mutant v2 and/or v3 fHbp proteins in immunogenic form, and administration of the vesicles can provide bactericidal antibody response as discussed in reference 155. The strain can also express a v1 fHbp, or a v1 fHbp can instead be provided as a separate recombinant protein in soluble form (and the v1 fHbp can be a wild-type or a mutant sequence e.g. mutated to disrupt its ability to bind to fH, as discussed above). The invention provides such strains, and also provides the vesicles which these strains release e.g. as purified from culture media after growth of the strains. A preferred v2 mutant for expression in these strains has a mutation at S32 and/or L123 as discussed herein, and a preferred v3 mutant for expression in these strains has a mutation at S32 and/or L126 as discussed herein. Thus vesicles prepared from meningococci expressing these v2 and v3 mutant fHbp sequences are particularly preferred immunogens for use in vaccines of the invention. A useful wild-type v2 sequence for mutagenesis in this way comprises SEQ ID NO: 51 or SEQ ID NO: 54 (comprising ΔG form SEQ ID NO: 55), and a useful wild-type v3 sequence for mutagenesis in this way comprises SEQ ID NO: 52.

Useful promoters for use in such strains include those disclosed in references 156 and 157. For instance, the promoter can be: (a) the promoter from a porin gene, preferably porA or porB, particularly from N. meningitidis; or (b) a rRNA gene promoter (such as a 16S rRNA gene), particularly from N. meningitidis. Where a meningococcal porin promoter is used, it is preferably from porA, and even more particularly a −10 region from a meningococcal porA gene promoter, and/or a −35 region from a meningococcal porA gene promoter (preferably wherein the −10 region and the −35 region are separated by an intervening sequence of 12-20 nucleotides, and wherein the intervening sequence either contains no poly-G sequence or includes a poly-G sequence having no more than eight consecutive G nucleotides). Where a rRNA gene promoter is used, it can comprise more particularly (i) a −10 region from a meningococcal rRNA gene promoter and/or (ii) a −35 region from a meningococcal rRNA gene promoter. It is also possible to use a hybrid of (a) and (b), for instance to have a −10 region from a porA promoter and a −35 region from a rRNA promoter (which can be a consensus −35 region). A useful promoter can thus be a promoter which includes either (i) a −10 region from a (particularly meningococcal) rRNA gene and a −35 region from a (particularly meningococcal) porA gene, or (ii) a −10 region from a (particularly meningococcal) porA gene and a −35 region from a (particularly meningococcal) rRNA gene.

If LOS is present in a vesicle it is possible to treat the vesicle so as to link its LOS and protein components (“intra-bleb” conjugation [150]).

### General

The term “comprising” encompasses “including” as well as “consisting” e.g. a composition “comprising” X may consist exclusively of X or may include something additional e.g. X+Y. References to “comprising” (or “comprises”, etc.) may optionally be replaced by references to “consisting of” (or “consists of”, etc.). The term “consisting essentially of” limits the scope of a claim to the specified materials or steps “and those that do not materially affect the basic and novel characteristic(s)” of the claimed invention.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

“Sequence identity” can be determined by the Smith-Waterman homology search algorithm as implemented in the MPSRCH program (Oxford Molecular), using an affine gap search with parameters gap open penalty=12 and gap extension penalty=1, but is preferably determined by the Needleman-Wunsch global alignment algorithm [158], using default parameters (e.g. with Gap opening penalty=10.0, and with Gap extension penalty=0.5, using the EBLOSUM62 scoring matrix). This algorithm is conveniently implemented in the needle tool in the EMBOSS package [159]. Where the application refers to sequence identity to a particular SEQ ID, the identity should be calculated over the entire length of that SEQ ID.

The term “fragment” in reference to polypeptide sequences means that the polypeptide is a fraction of a full-length protein. Such fragments may possess qualitative biological activity in common with the full-length protein, for example, a fragment may contain or encode one or more epitopes, such as immunodominant epitopes, that allow similar immune response to be raised to the fragment as to the full length sequence. Polypeptide fragments generally have an amino (N) terminus portion and/or carboxy (C) terminus portion deleted as compared to the native protein, but wherein the remaining amino acid sequence of the fragment is identical to the amino acid sequence of the native protein. Polypeptide fragments may contain, for example: about 8, 10, 12, 14, 16, 18, 20, 22, 24, 26, 28, 30, 32, 24, 26, 28, 40, 45, 50, 55, 60, 70, 80, 90, 100, 150, 200, 240, 241, 242, 243, 244, 245, 246, 247, 248, 249, 250, 251, 252, 253, 254, 255, 256, 257, 258, 259, 260, 261, 262 contiguous amino acids, including all integers in between, of a reference polypeptide sequence, for example between 50 and 260, 50 and 255, 50 and 250, 50 and 200, 50 and 150 contiguous amino acids of a reference polypeptide sequence. The term fragment explicitly excludes full length fHbp polypeptides and mature lipoproteins thereof.

After serogroup, meningococcal classification includes serotype, serosubtype and then immunotype, and the standard nomenclature lists serogroup, serotype, serosubtype, and immunotype, each separated by a colon e.g. B:4:P1.15:L3,7,9. Within serogroup B, some lineages cause disease often (hyperinvasive), some lineages cause more severe forms of disease than others (hypervirulent), and others rarely cause disease at all. Seven hypervirulent lineages are recognised, namely subgroups I, III and IV-1, ET-5 complex, ET-37 complex, A4 cluster and lineage 3. These have been defined by multilocus enzyme electrophoresis (MLEE), but multilocus sequence typing (MLST) has also been used to classify meningococci. The four main hypervirulent clusters are ST32, ST44, ST8 and ST11 complexes.

In general, the invention does not encompass the various fHbp sequences specifically disclosed in references 2, 3, 5, 6, 7, 160, 161, 162, 163, 164, 165, 166, and 167.

### BRIEF DESCRIPTION OF THE DRAWINGS

FIG. 1 shows the different sensitivity of fHbp variants to chymotrypsin cleavage. The arrow shows the position of full-length fHbp proteins.

FIG. 2 shows western blot analysis of v2 mutants. Lanes are: (1 & 2) Recombinant purified v2 wild type; (4) v2 wild type lysate; (5) mutant #1; (6) mutant #2; (7) mutant #4; (8) mutant #5; (9) mutant #7; (10) mutant #8; (11) mutant #12; (12) mutant #14 (13) mutant #15; (14) fHbp var2 NΔG-trx control i.e. v2 protein where N-terminal sequence GPDSDRLQQRR (SEQ ID NO: 37) is replaced by GSKDISS (SEQ ID NO: 38). Lanes 2-14 included chymotrypsin. M is molecular markers.

FIG. 3 shows further western blot analysis of v2 mutants. Lanes are: (1&2) mutant #3; (3&4) mutant #6; (5&6) mutant #9; (7&8) mutant #10; (9&10) mutant #13; (11&12) Δgono; (13&14) v2 wildtype; (15&16) mutant #22. Odd-numbered lanes are for proteins which were incubated without chymotrypsin, whereas even-numbered lanes were for proteins incubated with chymotrypsin. The ‘Δgono’ protein is a recombinant v2 where the N-terminal sequence (SEQ ID NO: 37) has been removed.

FIG. 4 shows further western blot analysis of v2 mutants. Lanes are: (l&2) mutant #11; (3&4) N-trx; (5-7) mutant #19; (8&9) mutant #20; (10&11) mutant #21. Lanes 2, 4, 7, 9 and 11 proteins which were incubated with chymotrypsin, whereas the other lanes had no chymotrypsin. The ‘N-trx’ protein is a recombinant v2 where the N-terminal sequence (SEQ ID NO: 37) is replaced by SEQ ID NO: 39.

FIG. 5 shows DSC results for wild-type and S58V/L149R mutant v2 fHbp. The C-terminal domain was unaffected by the mutation, but the Tm of the N-terminal domain was increased by >20° C. (marked with the arrow). The y-axis shows Cp (kcal/mol/° C.), and the x-axis show temperature (° C.).

FIG. 6 shows the SPR response of wild-type (solid) and mutant (dashed) v2 fHbp.

FIG. 7 shows the SPR response of v3 fHbp, either as wild-type (top) or with various mutations.

FIG. 8 shows DSC results for the triple fusion protein of SEQ ID NO: 48. The axes are as in FIG. 5.

### SPECIFIC EMBODIMENTS OF THE INVENTION

The invention provides the following specific numbered embodiments:

I. A mutant v3 or v2 fHbp which has increased stability relative to a wild-type fHbp and, preferably, also has lower affinity for human factor H than a wild-type fHbp;
for instance:
(A) a polypeptide comprising a mutant fHbp v2 amino acid sequence, wherein: (a) the amino acid sequence has at least 80% sequence identity to SEQ ID NO: 5 and/or comprises a fragment of SEQ ID NO: 5 which is at least 7 amino acids long and contains at least one of the residues listed in (b); but (b) the amino acid sequence differs from SEQ ID NO: 5 at one or more of the following residues: 32, 33, 39, 41, 69, 100, 113, 122, 123, 124, 125, 126, 127, 128, 151, 239, and/or 240; provided that:
   if the mutant fHbp v2 amino acid sequence includes a substitution only at residue 32, either this substitution is not with alanine or at least one further residue listed in (b) is substituted;
   if the mutant fHbp v2 amino acid sequence includes a substitution only at residue 113, either this substitution is not with alanine or at least one further residue listed in (b) is substituted.
   if the mutant fHbp v2 amino acid sequence includes a substitution only at residue 122, either this substitution is not with alanine or at least one further residue listed in (b) is substituted;
   if the mutant fHbp v2 amino acid sequence includes a substitution at residue 123, either this substitution is not with alanine or at least one further residue listed in (b) is substituted;
   if the mutant fHbp v2 amino acid sequence includes a substitution only at residue 124, either this substitution is not with alanine or at least one further residue listed in (b) is substituted;
   if the mutant fHbp v2 amino acid sequence includes a substitution only at residue 127, either this substitution is not with alanine or at least one further residue listed in (b) is substituted; and
   if the mutant fHbp v2 amino acid sequence includes a substitution only at residue 240, either this substitution is not with alanine or at least one further residue listed in (b) is substituted.
or (B) a polypeptide comprising a mutant fHbp v3 amino acid sequence, wherein: (a) the amino acid sequence has at least 80% sequence identity to SEQ ID NO: 17 and/or comprises a fragment of SEQ ID NO: 17 which is at least 7 amino acids long and contains at least one of the residues listed in (b); but (b) the amino acid sequence differs from SEQ ID NO: 17 at one or more of the following residues: 32, 33, 39, 41, 72, 103, 116, 125, 126, 127, 128, 129, 130, 131, 154, 242, and/or 243; provided that:
   if the mutant fHbp v3 amino acid sequence includes a substitution at residue 32, either this substitution is not with alanine or at least one further residue listed in (b) is substituted;
   if the mutant fHbp v3 amino acid sequence includes a substitution at residue 113, either this substitution is not with alanine or at least one further residue listed in (b) is substituted.
   if the mutant fHbp v3 amino acid sequence includes a substitution only at residue 125, either this substitution is not with alanine or at least one further residue listed in (b) is substituted;
   if the mutant fHbp v3 amino acid sequence includes a substitution at residue 126, either this substitution is not with alanine or at least one further residue listed in (b) is substituted;
   if the mutant fHbp v3 amino acid sequence includes a substitution at residue 127, either this substitution is not with alanine or at least one further residue listed in (b) is substituted;
   if the mutant fHbp v3 amino acid sequence includes a substitution at residue 130, either this substitution is not with alanine or at least one further residue listed in (b) is substituted; and
   if the mutant fHbp v3 amino acid sequence includes a substitution only at residue 243, either this substitution is not with alanine or at least one further residue listed in (b) is substituted.

2. The polypeptide of embodiment 1(B), wherein the amino acid sequence differs from SEQ ID NO: 17 by substitution at one or more of the residues listed in (b); for instance, where the substitution(s) are selected from the group consisting of: S32V; I33C; L39C; L41C; F72C; V103T; T116S; F125C; L126R; V127I; S128G or S128T; G129D; L130I; G131A; S154C; H242R; and E243H.

3. The polypeptide of embodiment 1(B) or embodiment 2, comprising more than one substitution at the residues listed in (b), and selected from groups 3A to 30 as noted above.

4. The polypeptide of embodiment 1(A), wherein the amino acid sequence differs from SEQ ID NO: 5 by substitution at one or more of the residues listed in (b); for instance, where the substitution(s) are selected from the group consisting of: S32V; V33C; L39C; L41C; F69C; V100T; I113S; F122C; L123R; V124I; S125G or S125T; G126D; L127I; G128A; S151C; H239R; and E240H.

5. The polypeptide of embodiment 1(A) or embodiment 4, comprising more than one substitution at the residues listed in (b), and selected from groups 2A to 20 as noted above.

6. The polypeptide of any of embodiments 1-5, also including one or more further mutation(s) which disrupt(s) the polypeptide's ability to bind to human factor H; for instance, in v2 including a substitution at one or more of R73, D203, E210, G228, S121, F122, L123, A192, E194, V199, I200, L201, T213, H215, F219, T231, and E240, or in v3 including a substitution at one or more of Q35, I178, L87, A88, L126, V127, V202, E213, T216, T234, V241, and E243.

7. A polypeptide comprising:
amino acid sequence SEQ ID NO: 44, optionally with 1, 2, 3, 4, or 5 single amino acid substitutions, deletions and/or insertions, wherein the polypeptide can elicit antibodies which bind to a meningococcal fHbp polypeptide consisting of the amino acid sequence of SEQ ID NO: 40 (for example, comprising amino acid sequence SEQ ID NO: 44 with 1, 2, or 3 single amino acid substitutions), but not mutated at residue V32 or R126;
amino acid sequence SEQ ID NO: 45, optionally with 1, 2, 3, 4, or 5 single amino acid substitutions, deletions and/or insertions, wherein the polypeptide can elicit antibodies which bind to a meningococcal fHbp polypeptide consisting of the amino acid sequence of SEQ ID NO: 2 (for example, comprising amino acid sequence SEQ ID NO: 45 with 1, 2, or 3 single amino acid substitutions), but not mutated at residue V32 or R123;
a fHbp v3 amino acid sequence, wherein the v3 amino acid sequence is identical to a v3 wild-type amino acid sequence except for a mutation at the amino acid position corresponding to Leu-126 of SEQ ID NO: 17, provided that the mutation is not a substitution to alanine (e.g. wherein the mutation is a substitution to arginine);
a fHbp v2 amino acid sequence, wherein the v2 amino acid sequence is identical to a v2 wild-type amino acid sequence except for a mutation at the amino acid position corresponding to Leu-123 of SEQ ID NO: 5, provided that the mutation is not a substitution to alanine (e.g. wherein the mutation is a substitution to arginine); or
amino acid sequence SEQ ID NO: 47, optionally with 1, 2, 3, 4, or 5 single amino acid substitutions, deletions and/or insertions, wherein the polypeptide can elicit antibodies which bind to each of: a meningococcal fHbp polypeptide consisting of the amino acid sequence of SEQ ID NO: 46; a meningococcal fHbp polypeptide consisting of the amino acid sequence of SEQ ID NO: 4; and a meningococcal fHbp polypeptide consisting of the amino acid sequence of SEQ ID NO: 40 (for instance, consisting of amino acid sequence SEQ ID NO: 48).

8. A plasmid or other nucleic acid comprising a nucleotide sequence encoding the polypeptide of any of embodiment 1 to 7.

9. A host cell transformed with the plasmid of embodiment 8; e.g. wherein the cell is a meningococcal bacterium, such as a meningococcal bacterium having down-regulation or knockout of mltA and also optionally has down-regulation or knockout of: (i) at least one gene involved in rendering the lipid A portion of LPS toxic, particularly of lpxl1; and/or (ii) at least one gene involved in capsular polysaccharide synthesis or export, particularly of synX and/or ctrA.

10. Membrane vesicles prepared from the host cell of embodiment 9, wherein the vesicles include a polypeptide of any one of embodiments 1 to 7.

11. An immunogenic composition comprising a polypeptide of any one of embodiments 1 to 7, or a vesicle of embodiment 10.

12. The composition of embodiment 11, further comprising a second polypeptide that, when administered to a mammal, elicits an antibody response that is bactericidal against meningococcus.

13. The composition of embodiment 11 or 12, further comprising (i) a conjugated capsular saccharide from N. meningitidis serogroup A, C, W135 and/or Y and/or (ii) a conjugated capsular saccharide from S. pneumoniae

14. A method for raising an antibody response in a mammal, comprising administering an immunogenic composition of any of embodiments 11 to 13.

### MODES FOR CARRYING OUT THE INVENTION

fHbp Mutations

The v2 fHbp is recognised as being unstable. To analyse the underlying structural reasons for this undesirable property, with a view to engineering the sequence to improve stability, the inventors analysed sequence alignments and 3D structures of fHbp polypeptides. One area of particular interest was the structural interface between the N-terminal and C-terminal domains [168].

The inventors identified the mutations explained in Table 1. Three of the positions identified for mutation overlap with references 24 and 25, but the invention does not encompass the polypeptides reported in the prior art i.e. where the polypeptides include substitutions solely at these positions by alanine. For instance, E240 can be substituted with histidine to match v1, and is ideally paired with substitution at residue H239 (mutants #1 and #11). Similarly, if F122 is substituted then it is preferably paired with substitution at S151, both with cysteine to permit formation of a disulfide bridge (mutant #10). Also, if L123 is substituted then it is can be substituted with arginine (rather than alanine), or it can be paired with substitution at other residues e.g. at S32 (see mutant #3), at S125 (see mutants #20 and #22), or with substitution at residues 124-128 (see mutant #12).

### Stability Studies

Unstable proteins tend to be less folded and for this reason prone to cleavage and degradation by proteases. FIG. 1 shows that v2 fHbp is more sensitive to chymotrypsin degradation than v1 and v3, and so this test can be used to assess stability of the mutant proteins.

For FIG. 1, wild-type fHbp v1, v2 and v3 were prepared at 0.5 mg/μL in 50 mM Tris-HCl, 150 mM NaCl, pH 8. Chymotrypsin was added at 1:100 (w/w) ratio. Samples were incubated at 24° C., 50 mL volume, no shaking. Samples were extracted and boiled for 0, 1, 3, or 6 hours; then run on 12% bis-Tris gel (MES buffer). The left-hand lane, marked *, indicates a sample incubated for 6 hours without protease.

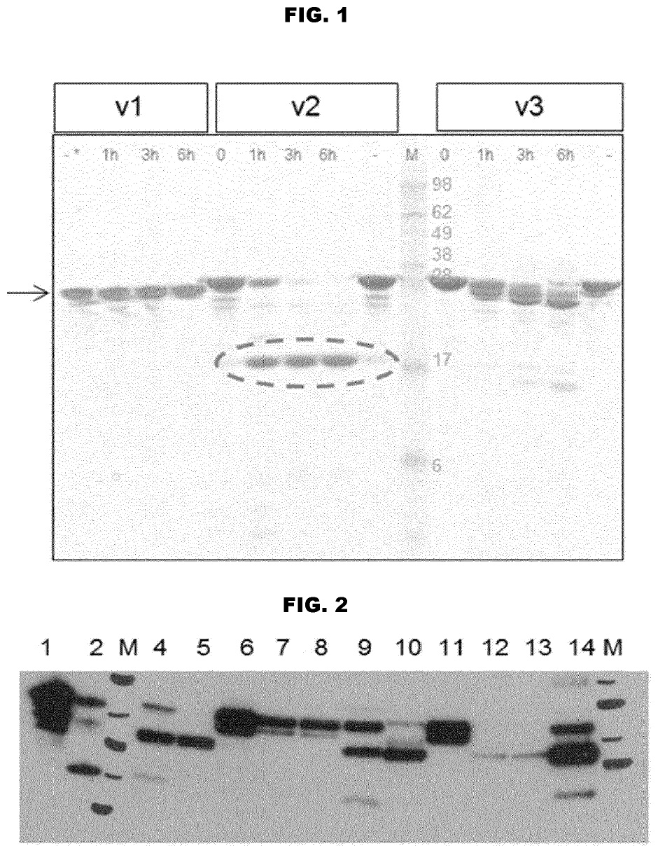

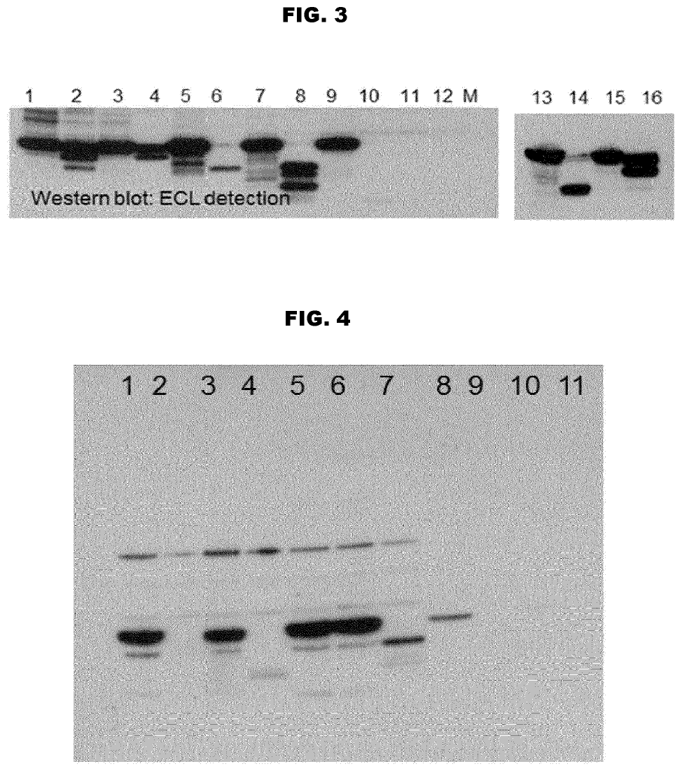

Cell lysates of E. coli expressing the recombinant proteins have been incubated with 1:100 w/w ratio chymotrypsin for 3 hours at 25° C. The degradation pattern has been analysed by Western blotting following the incubation with an immune polyclonal serum elicited in rabbit against all three fHbp variants. The presence of cleavage products at lower apparent molecular weight (FIGS. 2-4) is interpreted as an indication of instability, whereas persistence of a band corresponding to an apparent MW of ˜30 kDa is interpreted as an indication of increased stability. Mutants #1-6, #12 and #22 all showed increased resistance to chymotrypsin cleavage compared to the wild type v2.

DSC has been used as an independent approach to assess the effects of mutations on the stability of purified recombinant fHbp v2 proteins. Tₘ (melting temperature) measured by DSC corresponds to the temperature at which the analysed protein is 50% in the folded state and 50% in the unfolded state. Changes which stabilize the conformation of a protein will increase Tm, whereas destabilizing changes will decrease Tm. As seen in FIG. 3D of ref. 24, the DSC profile of wild-type v2 fHbp shows two Tm values: Tₘ₁ at ˜40° C., which corresponds to the melting temperature of the N-terminal domain, and Tₘ₂ at ˜80° C. corresponding to the melting temperature of the C-terminal domain. Values of Tₘ₁ and Tₘ₂ for analyzed mutants are shown in Table 1. Mutants #2, #4, #5, #12, #19 and #21 showed increased Tₘ of the N-terminal domain relative to the wild-type protein, and this effect was more marked for mutants #2, #4 and #12.

Size-exclusion chromatography (SEC) was used to assess the percentage of monomeric protein, and results are also shown in Table 1.

### Mutants #2, #3, and #4

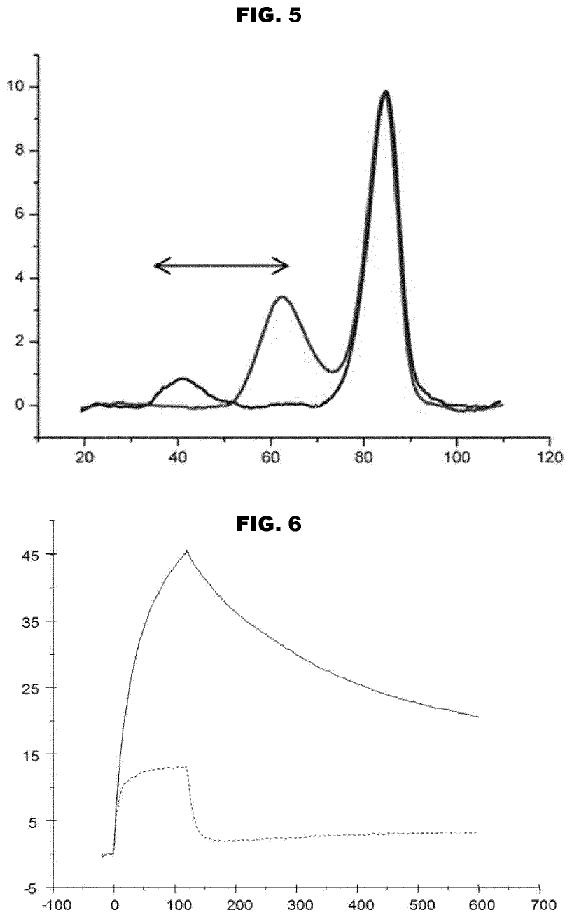

Mutant #3 (group 2B) gave the best overall results in the v2 stability studies. This protein (SEQ ID NO: 20) includes mutations at Ser-58 (S32 in SEQ ID NO: 5) and Leu-149 (L123 in SEQ ID NO: 5), with substitutions by Val and Arg, respectively. The mutant v2 protein (SEQ ID NO: 20, comprising SEQ ID NO: 45) was analysed by DSC and, compared to the wild-type sequence, the Tₘ of its N-terminus domain is >20° C. higher (FIG. 5).

In a serum bactericidal assay this v2 mutant could compete for binding to human antibodies which had been raised using a wild-type v2 sequence.

Although the S58V and L149R mutations had been introduced to improve stability, and did indeed achieve this goal, FIG. 6 shows that the mutant v2 polypeptide (dotted line) surprisingly showed much reduced binding to fH compared to the wild-type v2 sequence (solid line) when measured by surface plasmon resonance against immobilised fH. The S58V mutation on its own had little impact on fH binding, and the S58V/L149R double mutant showed higher fH binding than fHbp carrying only the L149R mutant.

When mutant #3 was further combined with the ‘E313A’ mutation in v2 there was a complete loss of fH binding as assessed by SPR.

The equivalent mutations were introduced into a v3 sequence (SEQ ID NO: 17), to give v3 mutant SEQ ID NO: 44. The effects of the individual S58V and L149R mutations on fH binding were studied in v3 (i.e. the v3 equivalents of v2 mutants #2 and #4). Thus, numbered according to SEQ ID NO: 17, mutation S32V or L126R was introduced into the v3 sequence. These two mutants were compared to two different wild-type v3 sequences, and also to the ‘E313A’ mutant which is known to disrupt fH binding in v3 [23].

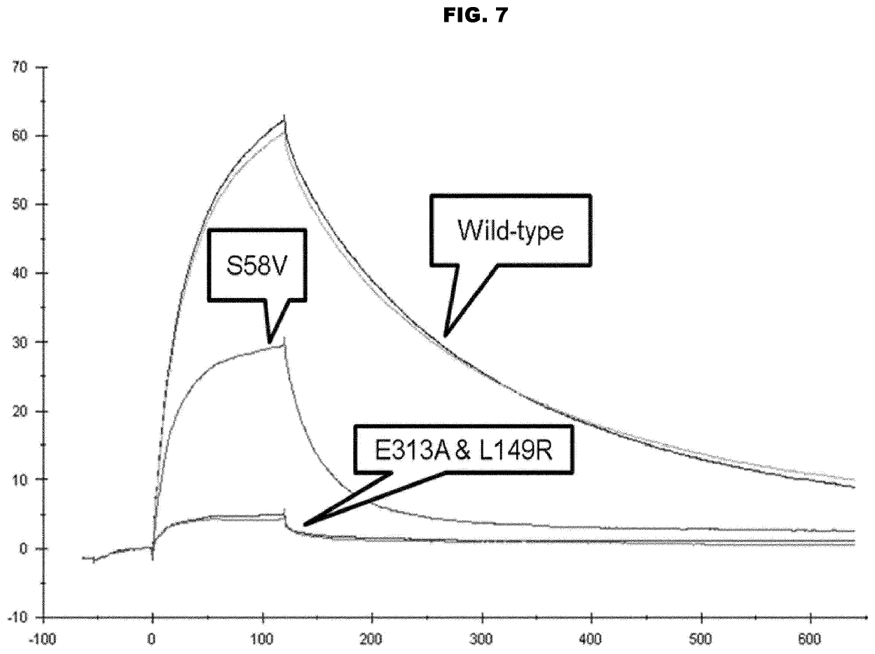

As shown in FIG. 7, wild-type v3 binds fH (top two lines). The S58V mutation, which was designed to improve stability, reduced the SPR peak value by about 2-fold. Most surprisingly, the L149R mutation (again, designed to improve stability) reduced fH affinity to a similar level to the known E313A mutant (bottom two lines).

The S58V and L149R mutations in v3 were also studied by DSC, and were found to increase the N-terminal Tm by 5.5° C. (S58V) or by 6.7° C. (L149R) relative to wild-type. The Tm of both mutants was higher than seen in the v2 double mutant (63.5° C.—see Table 1). The L149R v3 mutant also showed a higher Tm value for its C-terminal domain, whereas there was almost no shift here for the S58V v3 mutant. SPR showed that fH binding by mutant #2 was reduced by about a half, but for mutant #4 fH affinity was reduced to a similar level to the known E313A mutant (as also seen with v2). When the two mutations were combined (i.e. mutant #3) the Tₘ increase compared to wild-type was 11.2° C. When the ‘E313A’ mutation was added to mutant #3 fH binding was almost completely eliminated, although the T, of the N-terminal domain also decreased by 2.9° C. when compared to mutant #3 (while remaining 8.3° C. higher than v3 wild-type). The ‘E313A’ mutation alone was much less stable than wild-type, showing a Tm decrease of 6.3° C.

Thus mutations #2 and #4 can be used alone, or in combination (i.e. mutant #3), optionally with further mutations, to stabilise v2 or v3 fHbp but also to disrupt fH affinity.

A serum bactericidal assay was used for assessing the immunogenic efficacy of mutants #3 and #4 in v2 and v3. In addition, the ‘E313A’ mutant was also tested in v2 and v3, either alone or in combination with the #3 mutations. Wild-type v2 and v3 fHbp were also tested for comparison. The proteins were administered at 20 μg/dose with an aluminium hydroxide adjuvant and the resulting sera were tested for bactericidal activity against a panel of seven strains (four v2 strains, three v3 strains) including strains which express the same fHbp as the starting wild-type fHbp sequences (i.e. v2 sequence 2.16 and v3 sequence 3.42).

Results for the v2 proteins were as follows (SEQ ID is for the ΔG form; *=homologous fHbp):

| | SEQ | SBA against v2 strains | | | | SBA against v3 strains | | |
|---|---|---|---|---|---|---|---|---|
| Protein | ID | v2.16* | v2.19 | v2.21 | v2.24 | v3.42* | v3.28 | v3.30 |
| w.t. | 5 | 32768 | 32 | 32 | 1024 | >32768 | <16 | 32 |
| #4 | 21 | 32768 | <16 | <16 | 128 | 1024 | <16 | 16 |
| #3 | 45 | 4096 | 32 | <16 | <16 | >32768 | 128 | <16 |
| #3 + | 57 | 4096 | 16 | <16 | <16 | >32768 | <16 | <16 |
| E313A | | | | | | | | |
| E313A | 56 | 128 | 16 | <16 | <16 | 4096 | <16 | <16 |

Results for the v3 proteins were as follows:

| | SEQ | SBA against v3 strains | | | SBA against v2 strains | | | |
|---|---|---|---|---|---|---|---|---|
| Protein | ID | v3.42* | v3.28 | v3.30 | v2.16* | v2.19 | v2.21 | v2.24 |
| w.t. | 17 | >32768 | 512 | 1024 | 1024 | 32 | 64 | 256 |
| #4 | 53 | 1024 | <16 | 16 | <16 | 16 | <16 | 128 |
| #3 | 44 | 4096 | 32 | 16 | 4096 | <16 | 16 | 16 |
| #3 + | 43 | 256 | <16 | 16 | 64 | <16 | <16 | 16 |
| E313A | | | | | | | | |
| E313A | 41 | 4096 | 32 | 256 | 8192 | 32 | 32 | 128 |

### Combination of Mutants #2 and #12

Mutants #2 and #12 each showed improvements in v2 stability, so these two mutants were combined into a single fHbp (SEQ ID NO: 58, ΔG form). Compared to mutant #12 the N-terminal Tₘ of this combined mutant increased by a further 4.2° C., giving the highest Tₘ of any of the tested mutant v2 proteins. Furthermore, it showed a strongly reduced fH binding (SPR peak value reduced about 8×).

### Mutant Fusion Protein

Mutant v2 and v3 sequences were fused via a GSGGGG linker (SEQ ID NO: 50), also with a mutant v1 sequence, to give SEQ ID NO: 48. This sequence includes the S58V and L149R mutations for both v2 and v3, and the R41 S mutation [21] for vi. SEQ ID NO: 47 includes, from N-terminus to C-terminus: v2 mutant #3 (SEQ ID NO: 45); v3 mutant #3 (SEQ ID NO: 44); and v1 ‘R41S’ mutant (SEQ ID NO: 49), connected by the glycine-rich linker sequence, SEQ ID NO: 50. The fusion protein can conveniently be expressed by adding a N-terminus sequence of Met-[SEQ ID NO: 37]-, thus providing a mature protein SEQ ID NO: 48.

This fusion protein thus takes advantage of the observation that mutant #3 provides for both v2 and v3 a large increase in stability (Tₘ) and a large decrease in fH affinity. For v1 the R41 S mutation has little effect on thermal stability but strongly reduces fH binding.

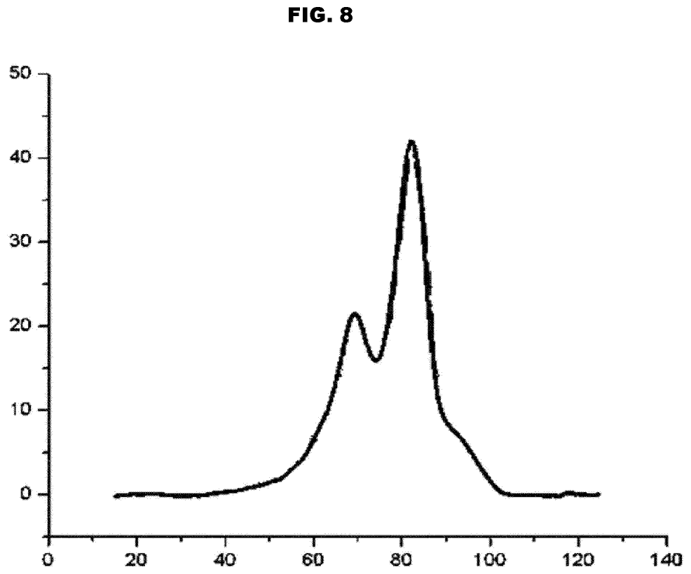

DSC studies of the triple fusion protein (FIG. 8) show that the three N-terminal transitions fall together in a broad peak centred at 68° C. The three C-termini transitions also fall together. UPLC showed that the protein was 94.9% pure, and HPLC analysis showed <1.5% oligomers.

### Mutant Proteins Expressed in ‘GMMA’ Membrane Vesicles

A v1 meningococcal strain was prepared with knockouts of mltA, lpxL1 and synX to provide a genetic background for hyper-expressing v2 and v3 fHbp lipoproteins under the control of the ‘ST2’ promoter [157] in a ‘GMMA’ vaccine. The v2 genes were integrated into the genome at the deleted lpxL1 locus whereas the v3 genes were integrated at the synX locus. In addition, the native v1 fHbp gene was deleted so that v2 and v3 could be studied without interference.

Mutants #3 and #4 were tested for v2, and mutant #4 was tested for v3. In addition, a strain with both of the v2 and v3 #4 mutants was prepared. For these bacteria fHbp expression and fH binding were assessed by FACS.

For strains expressing only v2 fHbp FACS showed that the various proteins were expressed at similar levels, at levels 2 logs higher than the background Δfhbp strain. In terms of fH binding, however, mutants #3 and #4 showed much less binding, with binding in mutant #4 being only slightly above background. These results mirror the SPR data obtained with recombinant v2 proteins.

For the strain expressing v3 mutant #4 FACS showed full expression of fHbp, but its fH binding was abolished (matching the fH binding seen with the ‘H222R’ mutation [19,25]).

For the strain expressing mutant #4 from v2 and v3, both fHbp proteins could be detected by FACS but fH binding was only slightly above that seen in the background Δfhbp strain.

Western blot analysis was used to test the stability of fHbp expression in these bacteria when growing in liquid culture for 6 days. Expression of mutant v2 proteins remained stable over time, even when v3 was co-expressed. Expression of mutant v3 proteins also remained stable, except in the strain expressing both the v2 and v3 mutant, where v3 expression declined over time.

It will be understood that the invention is described above by way of example only and modifications may be made whilst remaining within the scope and spirit of the invention.

### REFERENCES

[1] WO99/57280.
[2] Masignani et al. (2003) J Exp Med 197:789-799.
[3] Welsch et al. (2004) J Immunol 172:5605-15.
[4] Hou et al. (2005) J Infect Dis 192(4):580-90.
[5] WO03/063766.
[6] Fletcher et al. (2004) Infect Immun 72:2088-2100.
[7] Zhu et al. (2005) Infect Immun 73(10):6838-45.
[8] Cendron et al. (2011) Acta Crystallogr Sect F Struct Biol Cryst Commun. 67:531-5.
[9] Mascioni et al. (2009) J Biol Chem 284:8738-46.
[10] Pizza et al. (2008) Vaccine 26 Suppl 8:I46-8.
[11] Malito et al. (2013) PNAS USA 110:3304-9.
[12] Marshall et al. (2012) Pediatr Infect Dis J 31:1061-8.
[13] McNeil et al. (2013) Microbiol Mol Biol Rev 77:234-52.
[14] Serruto et al. (2012) Vaccine 30 Suppl2: B87-97.
[15] Scarselli et al. (2011) Sci Transl Med 3:91ra62.
[16] WO2011/051893.
[17] WO2010/046715.
[18] Schneider et al. (2009) Nature 458:890-5.
[19] WO2011/126863.
[20] Beernink et al. (2010) Clin Vaccine Immunol 17:1074-8.
[21] Beernink et al. (2011) J Immunol 186:3606-14.
[22] Rossi et al. (2013) Vaccine 31:5451-7.
[23] van der Veen et al. (2014) Infect Immun PMID 24379280.
[24] Johnson et al. (2012) PLoS Pathogen 8:e1002981.
[25] Pajon et al. (2012) Infect Immun 80:2667-77.
[26] Granoff et al. (2013) Clin Vaccine Immunol 20:1099-107.
[27] Beernink et al. (2008) Infect Immun 76:4232-40.
[28] Scarselli et al. (2009) J Mol Biol 386:97-108.
[29] Giuntini et al. (2012) PLoS One 7:e34272.
[30] Vu et al. (2012) Sci Rep 2:341.
[31] Faleri et al. (2013) FASEB J fj.13-239012.
[32] Johnson (2013) Arch Biochem Biophys 531:100-9.
[33] Bruylants et al. (2005) Current Medicinal Chemistry 12:2011-20.
[34] Veggi et al. (2012) Biochemistry 51:9384-93.
[35] WO2014/030003.
[36] Jongerius et al. (2013) PLoS Pathog 9(8): e1003528.
[37] Pizza et al. (2000) Science 287:1816-1820.
[38] WO2007/028408.
[39] http://pubmlst.org/neisseria/
[40] Budroni et al. (2011) PNAS USA 108:4494-99.
[41] Goldschneider et al. (1969) J. Exp. Med. 129:1307-26.
[42] Santos et al. (2001) Clinical and Diagnostic Laboratory Immunology 8:616-23.
[43] Frasch et ail. (2009) Vaccine 27S:B112-6.
[44] Gennaro (2000) Remington: The Science and Practice of Pharmacy. 20th edition, ISBN: 0683306472.
[45] WO03/009869.
[46] Vaccine Design . . . (1995) eds. Powell & Newman. ISBN: 030644867X. Plenum.
[47] Tettelin et al. (2000) Science 287:1809-1815.
[48] WO00/66741.
[49] Martin et al. (1997) J Exp Med 185(7):1173-83.
[50] WO96/29412.
[51] Perkins-Balding et al. (2003) Microbiology 149:3423-35.
[52] WO01/55182.
[53] WO01/38350.
[54] WO00/23595.
[55] Giuliani et al. (2006) Proc Natl Acad Sci USA. 103:10834-9.
[56] WO2004/032958.
[57] Costantino et al. (1992) Vaccine 10:691-698.
[58] Costantino et al. (1999) Vaccine 17:1251-1263.
[59] WO03/007985.
[60] Watson (2000) Pediatr Infect Dis J 19:331-332.
[61] Rubin (2000) Pediatr Clin North Am 47:269-285, v.
[62] Jedrzejas (2001) Microbiol Mol Biol Rev 65:187-207.
[63] Bell (2000) Pediatr Infect Dis J 19:1187-1188.
[64] Iwarson (1995) APMIS 103:321-326.
[65] Gerlich et al. (1990) Vaccine 8 Suppl:S63-68 & 79-80.
[66] Vaccines (1988) eds. Plotkin & Mortimer. ISBN 0-7216-1946-0.
[67] Del Guidice et al. (1998) Molecular Aspects of Medicine 19:1-70.
[68] Gustafsson et al. (1996) N. Engl. J. Med. 334:349-355.
[69] Rappuoli et al. (1991) TIBTECH 9:232-238.
[70] Sutter et al. (2000) Pediatr Clin North Am 47:287-308.
[71] Zimmerman & Spann (1999) Am Fam Physician 59:113-118, 125-126.
[72] McMichael (2000) Vaccine 19 Suppl 1:S101-107.
[73] Schuchat (1999) Lancet 353(9146):51-6.
[74] WO02/34771.
[75] Dale (1999) Infect Dis Clin North Am 13:227-43, viii.
[76] Ferretti et al. (2001) PNAS USA 98: 4658-4663.
[77] Kuroda et al. (2001) Lancet 357(9264):1225-1240; see also pages 1218-1219.
[78] Jones (2001) Curr Opin Investig Drugs 2:47-49.
[79] Ravenscroft et al. (1999) Vaccine 17:2802-2816.
[80] WO03/080678.
[81] Research Disclosure, 453077 (January 2002).
[82] EP-A-0372501.
[83] EP-A-0378881.
[84] EP-A-0427347.
[85] WO93/17712.
[86] WO94/03208.
[87] WO98/58668.
[88] EP-A-0471177.
[89] WO91/01146.
[90] Falugi et al. (2001) Eur J Immunol 31:3816-3824.
[91] Baraldo et al. (2004) Infect Immun 72(8):4884-7.
[92] EP-A-0594610.
[93] Ruan et al. (1990) J Immunol 145:3379-3384.
[94] WO00/56360.
[95] Kuo et al. (1995) Infect Immun 63:2706-13.
[96] Michon et al. (1998) Vaccine. 16:1732-41.
[97] WO02/091998.
[98] WO01/72337.
[99] WO00/61761.
[100] WO00/33882
[101] Lees et al. (1996) Vaccine 14:190-198.
[102] WO95/08348.
[103] U.S. Pat. No. 4,882,317
[104] U.S. Pat. No. 4,695,624
[105] Porro et al. (1985) Mol Immunol 22:907-919.s
[106] EP-A-0208375
[107] WO00/10599
[108] Gever et al. Med. Microbiol. Immunol, 165:171-288 (1979).
[109] U.S. Pat. No. 4,057,685.
[110] U.S. Pat. Nos. 4,673,574; 4,761,283; 4,808,700.
[111] U.S. Pat. No. 4,459,286.
[112] U.S. Pat. No. 4,965,338
[113] U.S. Pat. No. 4,663,160.
[114] U.S. Pat. No. 4,761,283
[115] U.S. Pat. No. 4,356,170
[116] WO02/09643.
[117] Katial et al. (2002) Infect Immun 70:702-707.
[118] WO01/52885.
[119] European patent 0301992.
[120] Bjune et al. (1991) Lancet 338(8775):1093-1096.
[121] Fukasawa et al. (1999) Vaccine 17:2951-2958.
[122] WO02/09746.
[123] Rosenqvist et al. (1998) Dev. Biol. Stand. 92:323-333.
[124] WO01/09350.
[125] European patent 0449958.
[126] EP-A-0996712.
[127] EP-A-0680512.
[128] WO02/062378.
[129] WO99/59625.
[130] U.S. Pat. No. 6,180,111.
[131] WO01/34642.
[132] WO03/051379.
[133] U.S. Pat. No. 6,558,677.
[134] WO2004/019977.
[135] WO02/062380.
[136] WO00/25811.
[137] Peeters et al. (1996) Vaccine 14:1008-1015.
[138] Vermont et al. (2003) Infect Immun 71:1650-1655.
[139] WO2006/081259.
[140] European patent 0011243.
[141] Fredriksen et al. (1991) NIPH Ann. 14(2):67-80.
[142] WO01/91788.
[143] WO2005/004908.
[144] WO98/56901.
[145] Claassen et al. (1996) 14(10):1001-8.
[146] WO99/10497.
[147] Steeghs et al. (2001) The EMBO Journal 20:6937-6945.
[148] Fisseha et al. (2005) Infect Immun 73:4070-80.
[149] WO2004/015099.
[150] WO2004/014417.
[151] WO2004/046177.
[152] WO2006/046143.
[153] Adu-Bobie et al. (2004) Infect Immun 72:1914-19.
[154] WO2011/036562.
[155] Koeberling et al. (2014) Vaccine 32:2688-95.
[156] WO2013/033398.
[157] WO2013/113917.
[158] Needleman & Wunsch (1970) J. Mol. Biol. 48, 443-453.
[159] Rice et al. (2000) Trends Genet 16:276-277.
[160] WO01/64920.
[161] WO03/020756.
[162] WO2004/048404.
[163] WO2004/094596
[164] WO2006/024954.
[165] WO2007/060548.
[166] WO2009/104097.
[167] WO2013/132452.
[168] Krissinel & Henrick (2007) J. Mol. Biol. 372:774-97.

TABLE 1

| | | Muta- | | SEQ | Chym | Tm1 | Tm2 | Mon |
|---|---|---|---|---|---|---|---|---|
| # | Residue(s)* | tion(s) | Notes | ID NO | ** | ° C. | ° C. | *** |
| 1 | H239 + E240 | R, H | Interface between N- and C-terminal domains. The aim is to mimic v1. | 18 | Yes | N/A | 83.5 | 34.29 |
| | | | An additional DSC transition was observed at 100.3° C., and some | | | | | |
| | | | aggregation was detected. | | | | | |
| 2 | S32 | V | N-terminal domain. Hydrophilic S32 side chain points into hydrophobic | 19 | Yes | 57.0 | 84.2 | 80.38 |
| | | | cavity. The aim is to increase hydrophobicity and stabilise the cavity. | | | | | |
| 3 | S32 + L123 | V, R | Mutants #2 + #4 | 20 | Yes | 63.5 | 83.84 | 76.1 |
| 4 | L123 | R | N-terminal domain. In v1 the reverse change decreased stability [11]. | 21 | Yes | 54.1 | 84.1 | 89.97 |
| 5 | S125 + G126 | G, D | N-terminal domain. The aim is to mimic v1 | 22 | Yes | 52.3 | 83.3 | 90.48 |
| 6 | V100 + S125 + | T, G, D | N-terminal domain. The aim is to mimic v1 | 23 | Yes | 52 | 83.7 | 86.41 |
| | G126 | | | | | | | |
| 7 | I113 | S | Surface loop of the N-terminal domain. Remove potential protease | 24 | No | ND | ND | ND |
| | | | cleavage site from surface | | | | | |
| 8 | V33 + L39 | C, C | Core of the N-terminal domain. Introduce a S-S bridge. Some | 25 | No | 55.9 | 85 | 28.71 |
| | | | aggregation was detected. | | | | | |
| 9 | L41 + F69 | C, C | Core of the N-terminal domain. Introduce a S-S bridge. | 26 | No | 46.4 | 84.4 | 33.5 + |
| | | | | | | | | 53.22 |
| 10 | F122 + S151 | C, C | Core of the N-terminal domain. Introduce a S-S bridge. | 27 | No | — | — | — |
| 11 | V100 + S125 + | T, G, D, | Mutants #1 + #6 | 28 | No | 47.9 | 82.2 | 85.1 |
| | G126 + H239 + | R, H | | | | | | |
| | E240 | | | | | | | |
| 12 | L123 − G128 | RIGDIA | N-terminal domain. The aim is to mimic v1 in the whole region of | 29 | Yes | 62.8 | 84.4 | 71.28 |
| | | | 123-128 | | | | | |
| 13 | V100 | T | Partial mutant #6 | 30 | No | 43 | 84.3 | 91.06 |
| 14 | L41 | C | Partial mutant #9. Some aggregation was detected. | 31 | No | — | 85 | 24.82 |
| 15 | F122 | C | Partial mutant #10. Some aggregation was detected. | 32 | No | — | 84.4 | 16.63 |
| 19 | S32 + S125 | V, T | N-terminal domain. Further increase hydrophobicity relative to #2 | 33 | No | 50.6 | 83.5 | 81.3 |
| 20 | S32 + S125 + | V, T, R | Combine #4 + #19 | 34 | No | — | — | — |
| | L123 | | | | | | | |
| 21 | S32 + S125 | V, G | N-terminal domain. Further increase hydrophobicity relative to #2 | 35 | No | 52.8 | 84 | 75.3 |
| 22 | S32 + S125 + | V, G, R | Combine #4 + #21 | 36 | Yes | — | — | — |
| | L123 | | | | | | | |
| 23 | S32 + L123 − | RIGDIAS | Combine #2 + #12 | 62 | Yes | 66.3 | 84.7 | — |
| | G128 | | | | | | | |

*Numbered according to SEQ ID NO: 5; add +26 to match SEQ ID NOs: 18 to 39.
** Resistance to chymotrypsin cleavage.
*** % monomeric form

1-15. (canceled)

16. A method for preventing or for protecting against Neisserial infection in a mammal comprising administering to the mammal an immunogenic composition comprising a fusion polypeptide, wherein the fusion polypeptide comprises a mutant v2 fHbp polypeptide and a mutant v3 fHbp polypeptide.

17. The method of claim 16, wherein the mutant v2 fHbp polypeptide comprises an amino acid sequence having at least 90% sequence identity to SEQ ID NO: 5 and includes a residue corresponding to residue 32 from SEQ ID NO: 5, wherein the amino acid sequence of the mutant v2 fHbp polypeptide differs from SEQ ID NO: 5 at residue 32 by the substitution S32V.

18. The method of claim 16, wherein the mutant v3 fHbp polypeptide comprises an amino acid sequence having at least 90% sequence identity to SEQ ID NO: 17 and includes a residue corresponding to residue 32 from SEQ ID NO: 17, wherein the amino acid sequence of the mutant v3 fHbp polypeptide differs from SEQ ID NO: 17 at residue 32 by the substitution S32V.

19. The method of claim 16, wherein the mutant v2 fHbp polypeptide further differs from SEQ ID NO: 5 by substitution at one or more of L123, V124, S125, G126, L127 and/or G128.

20. The method of claim 19, wherein the substitution(s) are selected from the group consisting of: L123R; V124I; S125G or S125T; G126D; L127I; and G128A.

21. The method of claim 16, wherein the mutant v2 fHbp polypeptide further comprises a L123 mutation.

22. The method of claim 21, wherein the L123 mutation is a L123R mutation.

23. The method of claim 17, wherein the mutant v3 fHbp polypeptide further differs from SEQ ID NO: 17 by substitution at one or more of S32, I33, L39, L41, F72, V103, T116, F125, L126, V127, S128, G129, L130, G131, S154, H242, and/or E243.

24. The method of claim 23, wherein the substitution(s) are selected from the group consisting of: S32V, I33C, L39C, L41C, F72C, V103T, T116S, F125C, L126R, V127I, S128G or S128T, G129D, L130I, G131A, S154C, H242R, and E243H.

25. The method of claim 16, wherein the mutant v3 fHbp polypeptide further comprises a L126 mutation.

26. The method of claim 23, wherein the L126 mutation is L126R.

27. The method of claim 16, wherein the immunogenic composition further comprises a polypeptide having the amino acid sequence of SEQ ID NO: 45; wherein SEQ ID NO: 45 is modified by up to 5 single amino acid substitutions, deletions and/or insertions; and wherein the 5 single amino acid substitutions, deletions and/or insertions do not include amino acid positions V32 and R123.

28. The method of claim 16, wherein the immunogenic composition further comprises a polypeptide having the amino acid sequence of SEQ ID NO: 44, wherein SEQ ID NO: 44 is modified by up to 5 single amino acid substitutions, deletions and/or insertions, and wherein the 5 single amino acid substitutions, deletions and/or insertions do not include amino acid positions V32 and R126.

29. The method of claim 16, wherein the mutant v2 fHbp polypeptide and the mutant v3 fHbp polypeptide are arranged in the order v2 fHbp polypeptide followed by v3 fHbp polypeptide from N- to C-terminus.

30. The method of claim 29, wherein a linker having the amino acid sequence of SEQ ID NO: 50 is between the mutant v2 fHbp polypeptide and the mutant v3 fHbp polypeptide.

31. The method of claim 16, further comprising (i) a conjugated capsular saccharide from N. meningitidis serogroup A, C, W135 and/or Y.

32. The method of claim 16, wherein the immunogenic composition comprises the fusion polypeptide in combination with one or more of (i) a NHBA polypeptide (ii) a NadA polypeptide and/or (iii) meningococcal outer membrane vesicles (OMVs).

33. The method according to claim 32, wherein the OMVs are from a serogroup B strain.

34. The method according to claim 32, wherein the OMVs are prepared from strain NZ98/254.

35. The method according to claim 32, wherein the immunogenic composition further comprises an aluminum hydroxide adjuvant.

36. The method according to claim 16, wherein said mammal is a human.


SEQUENCE LISTING (61 sequences)

SEQ ID NO: 1 - protein, 274 aa, Neisseria meningitidis
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 281 aa, Neisseria meningitidis
MNRTAFCCLSLTTALILTACSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 5 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 6 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 7 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 8 - protein, 174 aa, Neisseria meningitidis
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSAGVRVKF

SEQ ID NO: 9 - protein, 792 aa, Neisseria meningitidis
MKPLQMLPIAALVGSIFGNPVLAADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYNHHALGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWRVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKVKDAQYTVYENKGWGTPLQKKVKDYPWLNKSAYVFDMYGFYKPAKNLTLRAGVYNLFNRKYTTWDSLRGLYSYSTTNAVDRDGKGLDRYRAPGRNYAVSLEWKF

SEQ ID NO: 10 - protein, 591 aa, Neisseria meningitidis
MNKIYRIIWNSALNAWVVVSELTRNHTKRASATVKTAVLATLLFATVQASANNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGKSMMAIGGGTYRGEAGYAIGYSSISDGGNWIIKGTASGNSRGHFGASASVGYQW

SEQ ID NO: 11 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 12 - protein, 797 aa, Neisseria meningitidis
MKLKQIASALMMLGISPLALADFTIQDIRVEGLQRTEPSTVFNYLPVKVGDTYNDTHGSAIIKSLYATGFFDDVRVETADGQLLLTVIERPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSNQFNEQKFAQDMEKVTDFYQNNGYFDFRILDTDIQTNEDKTKQTIKITVHEGGRFRWGKVSIEGDTNEVPKAELEKLLTMKPGKWYERQQMTAVLGEIQNRMGSAGYAYSEISVQPLPNAETKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDVYGKAFDPRKASTSIKQYKTTTAGAGIRMSVPVTEYDRVNFGLVAEHLTVNTYNKAPKHYADFIKKYGKTDGTDGSFKGWLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGKTYDDNSSSATGGRVQNIYGAGNTHKSTFTNELRYSAGGAVTWLSPLGPMKFSYAYPLKKKPEDEIQRFQFQLGTTF

SEQ ID NO: 13 - protein, 180 aa, Neisseria meningitidis
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQR

SEQ ID NO: 14 - protein, 644 aa, Neisseria meningitidis
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 15 - protein, 327 aa, Neisseria meningitidis
ATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 16 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 17 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 18 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVRHIGIAGKQ

SEQ ID NO: 19 - protein, 273 aa, Neisseria meningitidis
MNRTAFCCLSLTAALILTACSSGGGGVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVRHIGIAGKQ

SEQ ID NO: 20 - protein, 273 aa, Neisseria meningitidis
MNRTAFCCLSLTAALILTACSSGGGGVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 21 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 22 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVGDLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 23 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVTALQIEKINNPDKIDSLINQRSFLVGDLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 24 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKSDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 25 - protein, 273 aa, Neisseria meningitidis
MNRTAFCCLSLTAALILTACSSGGGGVAADIGAGLADALTAPLDHKDKSLQSLTLDQSCRKNEKCKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 26 - protein, 273 aa, Neisseria meningitidis
MNRTAFCCLSLTAALILTACSSGGGGVAADIGAGLADALTAPLDHKDKSLQSLTLDQSVRKNEKLKCAAQGAEKTYGNGDSLNTGKLKNDKVSRCDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 27 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSCLVSGLGGEHTAFNQLPDGKAEYHGKAFSCDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 28 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVTALQIEKINNPDKIDSLINQRSFLVGDLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVRHIGIAGKQ

SEQ ID NO: 29 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFRIGDIAGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 30 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVTALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 31 - protein, 273 aa, Neisseria meningitidis
MNRTAFCCLSLTAALILTACSSGGGGVAADIGAGLADALTAPLDHKDKSLQSLTLDQSVRKNEKLKCAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 32 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSCLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 33 - protein, 273 aa, Neisseria meningitidis
MNRTAFCCLSLTAALILTACSSGGGGVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFLVTGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 34 - protein, 273 aa, Neisseria meningitidis
MNRTAFCCLSLTAALILTACSSGGGGVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVTGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 35 - protein, 273 aa, Neisseria meningitidis
MNRTAFCCLSLTAALILTACSSGGGGVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFLVGGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 36 - protein, 273 aa, Neisseria meningitidis
MNRTAFCCLSLTAALILTACSSGGGGVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVGGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 37 - protein, 11 aa, synthetic construct
  REGION 1..11: note = N-terminal sequence
GPDSDRLQQRR

SEQ ID NO: 38 - protein, 7 aa, synthetic construct
  REGION 1..7: note = N-terminal sequence
GSKDISS

SEQ ID NO: 39 - protein, 11 aa, synthetic construct
  REGION 1..11: note = N-terminal sequence
GSKDISSGGGG

SEQ ID NO: 40 - protein, 262 aa, Neisseria meningitidis
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 41 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQ

SEQ ID NO: 42 - protein, 250 aa, Neisseria meningitidis
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQ

SEQ ID NO: 43 - protein, 250 aa, Neisseria meningitidis
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQ

SEQ ID NO: 44 - protein, 250 aa, Neisseria meningitidis
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 45 - protein, 247 aa, Neisseria meningitidis
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 46 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 47 - protein, 757 aa, synthetic construct
  REGION 1..757: note = v2-v3-v1 mutant fusion
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 48 - protein, 769 aa, synthetic construct
  REGION 1..769: note = v2-v3-v1 mutant fusion
MGPDSDRLQQRRVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 49 - protein, 248 aa, Neisseria meningitidis
VAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 50 - protein, 6 aa, synthetic construct
  REGION 1..6: note = Linker
GSGGGG

SEQ ID NO: 51 - protein, 247 aa, Neisseria meningitidis
VAADIGARLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 52 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAELKADEKSHAVILGDTRYGGEEKGTYHLALFGDRAQEIAGSATVKIREKVHEIGIAGKQ

SEQ ID NO: 53 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 54 - protein, 273 aa, Neisseria meningitidis
MNRTAFCCLSLTTALILTACSSGGGGVAADIGARLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 55 - protein, 247 aa, Neisseria meningitidis; identical to SEQ ID NO: 51

SEQ ID NO: 56 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQ

SEQ ID NO: 57 - protein, 247 aa, Neisseria meningitidis
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQ

SEQ ID NO: 58 - protein, 247 aa, Neisseria meningitidis
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRIGDIAGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 59 - protein, 254 aa, Neisseria meningitidis
CSSGGGGVAADIGARLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 60 - protein, 254 aa, Neisseria meningitidis
CSSGGGGVAADIGARLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 61 - protein, 250 aa, Neisseria meningitidis; identical to SEQ ID NO: 44